SEQUENCE LISTING (2 sequences)

SEQ ID NO: 1 - DNA, 656 bp, Drosophila melanogaster
atggagaattctcgcacgcccacgaacaagaccaaaattacgcttaatcgcacgccaacgctaaaggagcgcagatggaacaccctgaaggtgaacacctccaacgtgcgatgctctactccgatctttggcaacttccgttcgcccaatctctcgcccatcgagaatatgggcacgaaggggaagagtccagtgtcgcccatgcggttcgctaccttcaagaaagtgccaacgaaggtgcatcccaagcagcagcagcagcagcagcatcagcactgccatcgcactcagtttaagcccccgccattcgtgctgcccaagccgcaggaggagatcatcgaaccggagcgagaaataaagagctgcagcagcccggatacctgttcggatgactcgaatatggagacctcactggcgttggagtcgcgtcgtcgttccatcaaagcatcgaaccactcgtacgtggttaaccatgccgccaatgtggaacagattctcatgcacatgggcctggagaactatgtgaccaatttcgaagaggctcacatcgatctggtggaactggcatccttggagcgtgctgatcttgttaaaatcggcctaaataccgatgaggattgcaaccgcatcatggatgtgctccacactctttaagt

SEQ ID NO: 2 - protein, 217 aa, Drosophila melanogaster
MENSRTPTNKTKITLNRTPTLKERRWNTLKVNTSNVRCSTPIFGNFRSPNLSPIENMGTKGKSPVSPMRFATFKKVPTKVHPKQQQQQQHQHCHRTQLKPPPFVLPKPQEEIIEPEREIKSCSSPDTCSDDSNMETSLALESRRRSIKASNHSYVVNHAANVEQILMHMGLENYVTNFEEAHIDLVELASLERADLVKIGLNTDEDCNRIMDVLHTL

(19) United States
(12) Patent Application Publication
(10) Pub. No.: US 2006/0074232 A1
(43) Pub. Date: Apr. 6, 2006

(54) Matrimony gene and protein

(75) Inventors: R. Scott Hawley, Overland Park, KS (US); Youbin Xiang, Overland Park, KS (US); Joseph J. Kramer, Kansas City, MO (US); David T. Harris, Somerville, MA (US)

Correspondence Address: POLSINELLI SHALTON WELTE SUELTHAUS P.C., 700 W. 47TH STREET, SUITE 1000, KANSAS CITY, MO, 64112-1802, US

(73) Assignee: Stowers Institute for Medical Research

(21) Appl. No.: 10/958,098

(22) Filed: Oct. 4, 2004

(51) Int. Cl. C12Q 1/68; C07H 21/02; C07K 14/47

(52) U.S. Cl. 536/23.1; 435/6; 435/69.1; 435/320.1; 435/325; 530/350

(57) ABSTRACT

The present invention is generally directed to Matrimony (Mtrm) nucleotide sequences, polypeptides expressed from the nucleotide sequences and methods employing the Mtrm nucleotide and polypeptide sequences. The Mtrm nucleotide sequences of the invention express a dosage dependent polypeptide that mediates achiasmate chromosome disjunction. In particular, reduction in the amount of the Mtrm polypeptide will cause nondisjunction in the achiasmate chromosomes.

### FIELD OF INVENTION

The present invention relates to a Matrimony (Mtrm) nucleotide sequence, which expresses a dosage dependent polypeptide that mediates achiasmate chromosome disjunction. In particular, reduction in the amount of the Mtrm polypeptide will cause nondisjunction of the achiasmate chromosomes.

### BACKGROUND OF INVENTION

It is known that during meiosis, the chromosome number is normally reduced by half. For example, in humans the chromosome number is reduced by half such that a human carrying 46 chromosomes produces sperm or eggs with 23 chromosomes. During the meiotic process, a spindle is formed with homologous chromosomes separating and moving away from one another. Proper separation of the chromosomes is important to ensure “normal” offspring. It is important to understand the mechanisms that control this process, as such mechanisms impact reproduction. It is especially desired to better understand genes and proteins which effect meiosis, as the isolated genes and proteins can be used to influence meiosis.

The accurate disjunction, or moving apart of chromosomes, during anaphase of mitotic or meiotic divisions of homologs during meiosis is accomplished by a series of highly coordinated processes beginning in meiotic prophase. In the chiasmate system of segregation, centromeres are oriented toward opposite poles by chiasmata, which restrict the movement of the chromosomes and serve to orient the centromeres toward the poles. Chiasmata are chromosomal sites where crossing over produces an exchange of homologous parts between non-sister chromatids. While most pairs of homologous chromosomes are chiasmate, achiasmate pairs, those lacking exchanges are sometimes formed.

Many organisms, including yeast, C. elegans, Homo sapien females and Drosophila melanogaster, employ a system of achiasmate chromosome segregation as a back-up system. This system is used in conjunction with chiasmata systems, and both are highly efficient when used in combination. But the achiasmate systems are less faithful compared to chiasmate segregation, and thus, nondisjunction occurs at a higher frequency for achiasmate homolog pairs. Nondisjunction is the failure of homologous chromosomes (in meiosis I) or sister chromatids (in meiosis II, or mitosis) to separate properly and to move to opposite poles. Nondisjunction results in one daughter cell receiving both, and the other daughter cell, none of the chromosomes or chromatids in question. In Drosophila females, the rate of achiasmate chromosome nondisjunction, as in humans, is usually close to or just less than 1%. A phenotypic example of nondisjunction would be Down's Syndrome in humans.

In Drosophila females, synaptonemal complex (SC) formation does not require exchange, so that nonexchange homologs pair and synapse normally. Association of nonexchange homologs is maintained in the presence of SC. At the end of pachytene, and concomitant with SC dissolution, the euchromatic arms of the chromosomes desynapse. Nonexchange homologs, nonetheless, remain associated by heterochromatic pairings, and heterochromatic homology is both necessary and sufficient to ensure centromere co-orientation. Although the ability of these heterochromatic associations to ensure stable disjunction will eventually depend on the Nod protein, Nod is not required to maintain heterochromatic associations from pachytene until nuclear envelope breakdown.

Currently, little is known about the mechanisms that underlie the maintenance of either homologous or heterologous heterochromatic associations in the oocyte nucleus. What is clear is that there is not a single heterochromatic chromocenter, but rather multiple smaller groups of associated pericentromeric heterochromatin. The composite of these smaller blocks of heterochromatic regions may vary among oocyte nuclei, but the X and 4th chromosome heterochromatin are almost always closely aligned. Little is known about the proteins that facilitate heterochromatic cohesion, and no mutants have been known that disrupts chromocentral organization.

Although haplo-insufficient genes are not uncommon, genes or regions that are haplo-insufficient for meiotic segregation in Drosophila are rare. There are but two known examples: Df(1)w^{rJ1}, a deficiency spanning the zeste-white region, and Df(3)sdb¹⁰⁵. Df(1)w^{rJ1} heterozygote females have frequent meiotic nondisjunction and reduced recombination. Identification of such regions, in particular genes, is important for better understanding nondisjunction and its causes. Haplo indicates an individual whose somatic or germ cells lack one member of a designated chromosomal region. As such, it is desired to have methods and compositions for studying haplo-insufficiency in achiasmate segregation, as well as identifying genes that cause haplo-deficiency in achiasmate. It is further desired to have constructs or tools for preventing such haplo-insufficiency.

Once a gene and its resultant phenotypic function are identified in Drosophila, this information can be used to identify homologs or orthologs. In particular, such a model can be applied to mammals, including humans, to identify and study related genes.

### SUMMARY OF INVENTION

The present invention relates to an isolated gene known as Matrimony (Mtrm), and the Mtrm protein. The protein or polypeptide expressed from the gene plays a crucial role in achiasmate chromosome disjunction. Reduction in the amount of Mtrm protein results in achiasmate nondisjunction. The present invention relates to an isolated wt Mtrm gene and protein. It also relates to the mutant heterozygote, which, when present, can cause haplo-insufficiency. Related to this are kits, which can be used to identify both mutants and wt sequences.

The Mtrm nucleotide sequence is listed as SEQ ID NO. 1. Other variations of the Mtrm nucleotide sequence may also be used. Homologs, orthologs, degenerate variants, homologous fragments, antisense sequences, and mutants of the Mtrm nucleotide sequence or SEQ ID NO 1 are also contemplated and available for use. Orthologous nucleotide sequences may be used, wherein the ortholog nucleotide sequence is derived from vertebrates or invertebrates. A transposable element which includes SEQ ID NO 1 or Mtrm, or any of the discussed nucleotide sequence can be formed. The transposable element is preferably a P-element; however, other transposable elements may be used. A heterozygote can be formed that has a wt Mtrm nucleotide sequence or allele and a mutant Mtrm nucleotide sequence or allele. This results in a haplo-insufficient chromosome having a mutant Mtrm sequence. Thus, the isolated nucleotide sequence for causing haplo-insufficiency during meiosis includes at least one Mtrm allele nucleotide sequence that is mutated to form either a heterozygous or homozygous mutant, which causes nondisjunction.

SEQ ID NO. 2 is a Mtrm protein. Variations of the Mtrm protein or polypeptide are also available for use. Homologous polypeptides, functional polypeptides, and amino acid sequences related to the Mtrm polypeptide or protein can be selected. Proteins expressed from the above genes or nucleotide sequences are also contemplated for use. The polypeptide can be derived from vertebrates or invertebrates. The Mtrm proteins can be used to form antibodies. The antibodies can be labeled and used to detect Mtrm protein

The Mtrm gene can also be used to identify organisms which have genes related to Mtrm.

Kits for detecting Mtrm mutants and wild types using a Mtrm nucleotide sequence, and a container are available. The hybridization kit for detecting a Mtrm mutant gene includes SEQ ID NO 1, or Mtrm nucleotide sequences, as well as homologous sequences thereof. Another kit for detecting a Mtrm gene uses PCR primers spanning a Mtrm family gene, a positive control, and sequencing products. A different kit has a container and an antibody derived from a Mtrm polypeptide, such as SEQ ID NO. 2, and homologs thereof.

### BRIEF DESCRIPTION OF DRAWINGS

FIG. 1 shows the effects of the deficiencies listed in Table 1 on the percentage of X and 4th chromosome nondisjunction;

FIG. 2A shows a cytogenetic map of region 66C, the breakpoints of the indicated deletions in reference to the polytene map are shown (FIG. 2B), denotes the known and predicted genes that lie within the limits defined by the deficiencies that are haplo-insufficient for achiasmate segregation (FIG. 2C), notes the site of insertion of the P(SUPor-P)KG08051 element relative to Mtrm (i.e., CG18543);

FIG. 3 illustrates PCR mapping of the endpoints of the deficiencies in 66C; whereby DNA obtained from embryos homozygous for the indicated deletions was amplified using various primer combinations corresponding to the positions noted, deficiency homozygotes were distinguished from siblings using a GFP marker carried on the balancer chromosome, all primer pairs spanned an interval of no more than 1 kb, the symbol “+” indicates a clear positive signal, while a “−” symbol indicates a failure to observe amplification; and,

FIG. 4 shows a metaphase I image of an FM7/X; Df(3L)T2-10 oocyte, the two chromosomes segregating to the upper pole are 4th chromosomes and the two segregating towards the lower pole are X-chromosomes, the X chromosome of the X (with two obviously separated blocks of heterochromatin) in FM7, the X on the right is its normal sequence homolog.

### DETAILED DESCRIPTION

The present invention relates to an isolated gene known as Matrimony (Mtrm), and the protein, or related polypeptide, expressed therefrom. The wild type (wt) protein expressed from the gene mediates achiasmate chromosome disjunction, at least in part, by ensuring that homologous centromeres properly co-orient (i.e., point to opposite poles). When a heterozygote containing a mutant Mtrm allele is formed, at least 50% of the amount of wt protein or polypeptide expressed is reduced, thereby causing achiasmate nondisjunction. As such, the present invention relates to both an isolated wt gene and an isolated mutant heterozygote, which, when present, can cause haplo-insufficiency. Related to this are kits, which can be used to identify both mutants and wt sequences, which impact haplo-insufficiency.

As explained, wt homozygous Mtrm expresses a polypeptide or protein that mediates disjunction of achiasmate chromosomes during meiosis. An example of the expressed Mtrm protein is SEQ ID NO 2. When a mutation occurs in either allele of the gene pair, the amount of wt protein expressed is reduced by at least 50%. The mutant allele will either express a mutant non-functional polypeptide, or protein, or nothing will be expressed. The mutant allele can cause nondisjunction in the achiasmate chromosomes.

The wt Mtrm gene is SEQ ID NO 1. Mtrm sequences, which are similar to SEQ ID NO 1, are also available for use. The similar Mtrm sequences will bind to SEQ ID NO 1 and express proteins that are functional equivalents to the protein expressed by SEQ ID NO 1. As such, related to SEQ ID NO 1 are isolated Mtrm sequences that are homologous and express a protein, such as SEQ ID NO 2. The protein of SEQ ID NO 2 is expressed by SEQ ID NO 1. The homologous Mtrm sequences function in the same way in that they promote heterochromatic association. A number of nucleotide sequences that express a functional Mtrm polypeptides may be used herewith. The Mtrm nucleotide sequences are initially isolated from Drosophila, but may be obtained from a number of other sources.

In certain aspects of the invention, accordingly, the Mtrm nucleotide sequence will be a sequence that encodes a polypeptide functionally equivalent to the Mtrm polypeptide having SEQ ID NO. 2. Generally speaking, the Mtrm protein mediates achiasmate chromosome disjunction, at least in part, by ensuring that homologous centromeres properly co-orient (i.e., point to opposite poles). Methods for determining whether a subject polypeptide performs this function are detailed in the examples. In one embodiment, the isolated nucleotide sequence will encode a polypeptide having the amino acid sequence of SEQ ID NO. 2 or of a fragment of SEQ ID NO. 2. Typically the fragment is at least 10 amino acid residues in length, more typically at least 15 residues in length, and even more typically is 25 to 50 amino acid residues in length. The fragment may be used for a variety of purposes including as an antigenic tool for the production of antibodies to a polypeptide having SEQ ID NO. 2.

In another embodiment, the isolated nucleotide sequence will encode a polypeptide having the amino acid sequence of SEQ ID NO. 2 or with conservative amino acid substitutions of SEQ ID NO. 2. The number of conservative amino acid substitutions can and will vary depending on the embodiment, but will range from approximately about 0 to about 100 conservative amino acid substitution. In a more preferable embodiment, the number of conservative amino acid substitutions will range from approximately about 0 to about 75, more preferably from about 0 to about 50 and even more preferably from about 0 to about 25. Because the amino acid substitutions are conservative, the subject polypeptide will generally possess the biological function of a polypeptide having SEQ ID NO. 2. Methods for determining whether a given amino acid substitution is conservative are described herein.

In still another embodiment, the isolated nucleotide sequence will encode a polypeptide that has an amino acid sequence that is at least 50% identical to the amino acid sequence of SEQ ID NO. 2. More typically, however, the isolated nucleotide sequence will encode a polypeptide that has an amino acid sequence that is at least 75% identical to the amino acid sequence of SEQ ID NO. 2 and even more typically, 90% identical to the amino acid sequence of SEQ ID NO. 2. In a particularly preferred embodiment, the nucleotide sequence will encode a polypeptide that has an amino acid sequence that is at least 95%, and even more preferably, 99% identical to the amino acid sequence of SEQ ID NO.2. In each of these embodiments, the isolated nucleotide sequence will preferably encode a polypeptide that facilitates the proper segregation of achiasmate chromosomes and is required in two doses for that function, as detailed herein.

In other aspects of the invention, the isolated nucleotide sequence will be substantially similar in sequence to SEQ ID NO. 1. In one embodiment, the isolated nucleotide sequence will be a sequence that is at least 50% identical to SEQ ID NO. 1. In still another embodiment, the isolated nucleotide will be a sequence that is at least 75% and even more typically, at least 80 to 90% identical to SEQ ID NO. 1. In a more preferred embodiment, the isolated nucleotide will be a sequence that is at least 95% and even more preferably, at least 99% identical to SEQ ID NO. 1. In each of these embodiments, the isolated nucleotide sequence will preferably encode a polypeptide that is able to mediate the disjunction of achiasmate chromosomes in a dose dependent manner, as detailed herein. Equally, in each embodiment, the isolated nucleotide sequence may be employed as a hybridization probe.

The invention also encompasses the use of nucleotide sequences other than SEQ ID NO. 1 that encode Mtrm polypeptides having the structure and function described herein. Typically, these nucleotide sequences will hybridize under stringent hybridization conditions (as defined herein) to all or a portion of the nucleotide sequence represented by SEQ ID NO. 1 or its complement. The hybridizing portion of the hybridizing nucleic acids is usually at least 15 (e.g., 20, 25, 30, or 50) nucleotides in length. The hybridizing portion of the hybridizing nucleic acid is at least 80%, preferably, at least 90%, and is more preferably, at least 95% identical to the sequence of a portion or all of a nucleic acid sequence encoding a Mtrm polypeptide suitable for use in the present invention, or its complement.

Hybridization of the oligionucleotide probe to a nucleic acid sample, such as SEQ ID NO. 1, is typically performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming at 1% mismatching results in a 1° C. decrease in the Tm, the temperature of the final wash in the hybridization reaction is reduced accordingly. For example, if sequences have greater than 95% identity with the probe is sought, the fmal temperature is approximately decreased by 5° C. In practice, the change in Tm can be between 0.5 and 1.5° C. per 1% mismatch. Stringent conditions involve hybridizing at 68° C. in 5×SSC/5× Denhardt's solution/1.0% SDS, and washing in 0.2×SSC/0.1% SDS at room temperature. Moderately stringent conditions include washing in 3×SSC at 42° C. The parameters of salt concentration and temperature can be varied to achieve the optimal level of identity between the probe and SEQ ID NO. 1. Additional guidance regarding such conditions is readily available in the art, for example, by Sambrook et al., 1989, Molecular Cloning, A Laboratory Manual, Cold Spring Harbor Press, N.Y.; and Ausubel et al., (eds.), 1995, Current Protocols in Molecular Biology, (John Wiley & Sons, N.Y.) at Unit 2.10.

The Drosophila Mtrm sequences disclosed herein can be used to isolate orthologs that can be used as part of a kit or which can be further studied to better understand nondisjunction in other organisms. The orthologs can also be used to form P-elements. Orthologous sequences can be used in the same applications as the Mtrm sequences.

The wt Mtrm gene is actually a pair of genes, with one copy of the gene located on each homolog. The alleles are homologous in the wt and promote disjunction. In the mutant, one of the two alleles is a mutant, also known as a heterozygote. In Drosophila, the Mtrm genes are found on chromosome 3. A heterozygote or homozygote may be formed in an organism by causing a mutation to one or both copies of the Mtrm gene. Alternatively, one or both copies of the Mtrm gene may be replace with a mutant copy. In either case, the heterozygote or homozygote may be utilized as a research tool to study chromosome non disjunction in an organism of interest.

Related to the isolated Mtrm gene and SEQ ID NO 1 are isolated-homologs, orthologs, degenerate variants, antisense sequences, and any other related sequences. Mutant sequences can be formed from the isolated wt Mtrm sequence. These mutants can be used to transfect a host to form heterozygote and homozygote mutants. A mutation can be made using a number of a variety of known processes, as long as at least one allele expresses a nonfunctional protein. The wt sequences can be mutagenized and used to express a mutant polypeptide or protein. Fragments of SEQ ID NO 1 or the Mtrm gene can be mutagenized to form mutant fragments. The mutant fragments should be at least 50% homologous, more preferably the fragments are 60%, 75%, or 90% homologous. Fragments homologous to the wt gene can also be isolated and used in kits, with the fragments at least 50% homologous, more preferably the fragments are 60%, 75%, or 90% homologous. The selected fragments should bind to wt or homologous Mtrm genes or nucleotide sequences. The length of the fragments can and will vary depending upon the embodiment, but generally the fragment length will be from 10 to about 100 nucleotides, and more typically will be at least 15 nucleotides in length.

Functionally, cytological studies of prometaphase and metaphase I in Mtrm heterozygotes demonstrate that achiasmate chromosomes appear to be randomly positioned on either side of the metaphase mass with respect to the position of the homolog. This implies that the presence of only a single copy of Mtrm disrupts the ability of achiasmate chromosomes to properly segregate to opposite poles. As such, this illustrates that Mtrm is dosage dependent.

Any of the above nucleotide sequences can be isolated and amplified, which is desired for use in kits and various studies. In particular, the Mtrm gene, or gene fragment, can be isolated and amplified. Amplification can be accomplished using well known techniques and methods. The gene can then be mutagenized. Any of a variety of methods can be used to form a mutant, including deletion, frame shift, point, or any of a variety of mutations, as long as the expressed protein is non-functional.

The nucleotide sequences can be used to form vectors for use in transfecting various types of cells. Any of a variety of vectors can be selected dependent upon the cell or species to be transfected, as well as the use of the vector.

Vectors are used to deliver the selected Mtrm nucleotide sequence to a host. The vectors will include RNA and DNA viruses, plasmids, and transposable elements. Transposable elements, specifically P-elements, can be formed from the isolated nucleotide sequences. The P-elements are used to study changes in the transfected species. In addition to the P-elements, Class II transposable elements may be used, such as minos, mariner, and piggybac. Transfection can be accomplished by injecting the vector into a blastocyte. The blastocyte will gestate and develop into a mature adult, with the mutant or foreign gene incorporated into the genome. A marker can be included in the vector. The mutant can be made conditional, if desired.

Transfected cells can be formed which include a transgenic Mtrm gene or mutant Mtrm gene to form the heterozygote. Thus, transfected cells can be formed that include a Mtrm mutant. The cells can be transfected using a variety of known techniques. The technique selected will depend upon the type of cell to be transfected. Related to vector transfection, transgenic animals can be formed.

The wt protein expressed from the Mtrm gene is SEQ ID NO 2. Proteins that maintain hetachromatic association and promote disjunction can be substituted for the Mtrm protein, so that proteins that are similar and have the same function may be used. The isolated wt Mtrm gene and protein can be used in a variety of different kits and systems. As mentioned, the Mtrm gene and protein can be used to identify orthologous or similar nucleotide sequences and polypeptides or proteins found in other species. The Mtrm sequences can also be used to identify genes and proteins that have a similar function. The genes and proteins can also be used to develop compositions that correct mutant Mtrm genes or proteins. Conversely, mutant Mtrm sequences can be used to promote nondisjunction.

Any of a variety of polypeptides or proteins related to SEQ ID NO 2 may be used in the practice of the invention. Depending upon the embodiment, amino acid sequences or polypeptides, which include a portion of the Mtrm protein may be used. Polypeptides or proteins that have substantially the same function as the Mtrm protein can be used. Moreover, the polypeptides can be of a variety of sizes. Also, mutant Mtrm proteins may also be of use. The mutant proteins may have a similar binding site but different functionality.

In one embodiment, the subject polypeptide will specifically bind to an antibody that binds specifically to a polypeptide having SEQ ID NO.2. Generally speaking, the polypeptides selected in this embodiment will have a substantially similar biological function to a Mtrm protein, such as the ability to mediate the disjunction of achiasmate chromosomes in a dosage dependent manner.

In yet another embodiment, the polypeptide will have an amino acid sequence that is at least 50% identical to SEQ ID NO.2. But in more preferable embodiments, the subject polypeptide will be at least 75% identical and even more typically at least 80 to 90% identical in amino acid sequence to SEQ ID NO.2. In a particularly preferred embodiment, the subject polypeptide will have an amino acid sequence that is at least 95% and more typically 99% identical to SEQ ID NO. 2. In each embodiment, the selected polypeptide will generally have a substantially similar biological activity to a Mtrm protein.

In a further embodiment of the invention, the polypeptide has an amino acid sequence that comprises SEQ ID NO. 2 with 0 to 100 conservative amino acid substitutions. In an alternative embodiment, the polypeptide may have from 0 to 50 conservative amino acid substitutions. In still other alternative embodiments, the polypeptide will have from 0 to 25 conservative amino acid substitutions or from 0 to 10 conservative amino acid substitutions. Because the amino acid substitutions are conservative in nature, the subject polypeptide will typically possess substantially the same biological activity as a polypeptide having SEQ ID NO.2.

In yet another embodiment, the subject polypeptide is an immunogenic polypeptide which comprises at least 10 consecutive amino acid residues of SEQ ID NO. 2. More typically, the immunogenic polypeptide will have at least 15 or 20 consecutive amino acid residues, but may have more than 25 consecutive amino acid residues of SEQ ID NO. 2. Among other uses, the polypeptides of this embodiment may be employed to produce antibodies to SEQ ID NO. 2.

A number of methods may be employed to determine whether a particular polypeptide possesses substantially similar biological activity relative to the Mtrm polypeptide having SEQ ID NO 2. Specific activity or function may be determined by convenient in vitro, cell-based, or in vivo assays, such as, in vitro binding assays. Binding assays encompass any assay where the molecular interaction of a subject polypeptide with a binding target is evaluated. The binding target may be a natural binding target such as a regulating protein or a non-natural binding target such as a specific immune protein such as an antibody, or a specific agent such as those identified in screening assays. In order to determine whether a particular polypeptide has the ability to mediate the disjunction of achiasmate chromosomes in a dosage dependent manner, the procedures detailed in the examples may be followed.

In addition to having a substantially similar biological function, as detailed above, a subject polypeptide suitable for use in the invention will also typically share substantial sequence identity to a Mtrm polypeptide and SEQ ID NO 2. In determining whether a polypeptide is substantially identical to Mtrm polypeptide or nucleotide sequence, sequence similarity may be determined by conventional algorithms, which typically allow introduction of a small number of gaps in order to achieve the best fit. In particular, “percent homology” of two polypeptides or two nucleic acid sequences is determined using the algorithm of Karlin and Altschul (Proc. Natl. Acad. Sci. USA 87:2264-2268, 1993). Such an algorithm is incorporated into the NBLAST and XBLAST programs of Altschul et al. (J. Mol. Biol. 215:403-410, 1990). BLAST nucleotide searches may be performed with the NBLAST program to obtain nucleotide sequences homologous to a nucleic acid molecule of the invention. Equally, BLAST protein searches may be performed with the XBLAST program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., XBLAST and NBLAST) are employed. See http://www.ncbi.nlm.nih.gov for more details.

Mtrm polypeptides suitable for use in the invention are isolated or pure. An “isolated” polypeptide is unaccompanied by at least some of the material with which it is associated in its natural state, preferably constituting at least about 0.5%, and more preferably, at least about 5% by weight of the total polypeptide in a given sample. A pure polypeptide constitutes at least about 90%, preferably, 95% and even more preferably, at least about 99% by weight of the total polypeptide in a given sample.

The Mtrm polypeptide may be synthesized, produced by recombinant technology, or purified from cells. In one embodiment, the Mtrm polypeptide of the present invention may be obtained by direct synthesis. In addition to direct synthesis, the subject polypeptides can also be expressed in cell and cell-free systems (e.g. Jermutus L, et al., Curr Opin Biotechnol. October 1998; 9(5):534-48) from encoding polynucleotides, such as from SEQ ID NO 1 (as described below) or naturally-encoding polynucleotides isolated with degenerate oligonucleotide primers and probes generated from the subject polypeptide sequences (“GCG” software, Genetics Computer Group, Inc, Madison Wis.) or polynucleotides optimized for selected expression systems made by back-translating the subject polypeptides according to computer algorithms (e.g. Holler et al. (1993) Gene 136, 323-328; Martin et al. (1995) Gene 154, 150-166). In other embodiments, any of the molecular and biochemical methods known in the art are available for biochemical synthesis, molecular expression and purification of the Mtrm polypeptides, see e.g. Molecular Cloning, A Laboratory Manual (Sambrook, et al. Cold Spring Harbor Laboratory), Current Protocols in Molecular Biology (Eds. Ausubel, et al., Greene Publ. Assoc., Wiley-Interscience, New York).

Yet a further aspect of the invention encompasses the use of Mtrm polypeptides or proteins to produce antibodies. Antibodies to any of the polypeptides suitable for use in the invention may be generated using methods that are well known in the art. Such antibodies may include, but are not limited to, polyclonal, monoclonal, chimeric, and single chain antibodies, Fab fragments, and fragments produced by a Fab expression library.

For the production of antibodies, various hosts including goats, rabbits, rats, mice, humans, and others may be immunized by injection with a subject polypeptide that has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to a selected polypeptide have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of the selected polypeptide's amino acid may be fused with those of another protein, such as KLH, and antibodies to the chimeric molecule may be produced.

Monoclonal antibodies to a polypeptide may be prepared using a technique that provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:3142; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-45). Alternatively, techniques described for the production of single chain antibodies may be adapted, using methods known in the art, to produce a Mtrm polypeptide-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments that contain specific binding sites for Mtrm polypeptides may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between the polypeptide and its specific antibody. A two-site, monoclonal-based immunoassay utilizing monoclonal antibodies reactive to two non-interfering polypeptide epitopes is generally used, but a competitive binding assay may also be employed.

Various methods such as Scatchard analysis in conjunction with radioimmunoassay techniques may be used to assess the affinity of antibodies for the subject polypeptide. Affinity is expressed as an association constant, Kₐ, which is defined as the molar concentration of polypeptide-antibody complex divided by the molar concentrations of free antigen and free antibody under equilibrium conditions. The Kₐ is determined for a preparation of polyclonal antibodies, which are heterogeneous in their affinities for multiple polypeptide epitopes, represents the average affinity, or avidity, of the antibodies for the particular polypeptides. The Kₐ is determined for a preparation of monoclonal antibodies, which are monospecific for a particular polypeptide epitope, represents a true measure of affinity. High-affinity antibody preparations with Kₐ ranging from about 10⁹ to 10¹² L/mole are preferred for use in immunoassays in which the polypeptide-antibody complex must withstand rigorous manipulations. Low-affinity antibody preparations with Kₐ ranging from about 10⁶ to 10⁷ L/mole are preferred for use in immunopurification and similar procedures that ultimately require dissociation of polypeptides, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparation for certain downstream applications. For example, a polyclonal antibody preparation containing at least 1-2 mg specific antibody/ml, preferably 5-10 mg specific antibody/ml, is generally employed in procedures requiring precipitation of a subject polypeptide-antibody complex. Procedures for evaluating antibody specificity, titer, and avidity, and guidelines for antibody quality and usage in various applications, are generally available. (See, e.g., Catty, supra, and Coligan et al. supra.)

Generally speaking, the antibodies of the invention may be utilized in a variety of applications such as for protein purification or for therapeutic uses. By way of example, the antibodies can be used to bind the wt Mtrm protein and, thereby cause nondisjunction in achiasmate chromosomes. Alternatively, the antibodies are also used as tools to mark the presence of the Mtrm protein. By way of example, antibodies of the present invention may be utilized in a method to detect a Mtrm heterozygote, such as in an oocyte. The marker antibodies typically will include a marker, such as a fluorescent marker, and will bind to the wt protein.

In other aspects of the invention, diagnostic methods are provided. One preferred embodiment employs a test compound to diagnose a Mtrm heterozygote. Examples of suitable test compounds employed in the diagnostic methods of the invention include antibodies, oligonucleotides, proteins (e.g., receptors), or small molecules, which may be produced as described above or in accordance with methods generally known in the art.

In one embodiment, as detailed above, antibodies that specifically bind Mtrm polypeptides may be used for the diagnostic methods of the invention. For example, the antibodies may be employed to detect Mtrm mediated achiasmate chromosome nondisjunction. Typically, the test will be performed on an oocyte. Antibodies useful for diagnostic purposes may be prepared in the same manner as described above. Diagnostic assays for Mtrm polypeptides include methods that utilize the antibody and a label to detect Mtrm polypeptides in body fluids or in extracts of cells or tissues. The antibodies may be used with or without modification, and may be labeled by covalent or non-covalent attachment of a reporter molecule. A wide variety of reporter molecules are known in the art and may be used.

In another embodiment of the invention, the polynucleotides encoding Mtrm polypeptides may be used for diagnostic purposes. The polynucleotides that may be used include oligonucleotide sequences, complementary RNA and DNA molecules, and PNAs. Generally speaking, the diagnostic assay may be used to determine absence, presence, and excess expression of Mtrm polypeptides, and to monitor regulation of Mtrm polypeptide levels during therapeutic intervention, if appropriate.

In one aspect, hybridization with PCR probes that are capable of detecting polynucleotide sequences, including genomic sequences, encoding Mtrm polypeptides or closely related molecules may be used to identify nucleic acid sequences that encode Mtrm polypeptides. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding Mtrm polypeptides, allelic variants, or related sequences.

In a particular aspect, the nucleotide sequences encoding Mtrm polypeptides may be useful in assays that detect Mtrm heterozygotes. The nucleotide sequences encoding Mtrm polypeptides may be labeled by standard methods and added to a fluid or tissue sample, such as an oocyte, from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding Mtrm polypeptides in the sample indicates aberrant Mtrm expression, which may result in achiasmate chromosome nondisjunction.

Additional diagnostic uses for oligonucleotides designed from the sequences encoding Mtrm polypeptides may involve the use of PCR. These oligomers may be chemically synthesized, generated enzymatically, or produced in vitro. Oligomers will preferably contain a fragment of a polynucleotide encoding Mtrm polypeptides, or a fragment of a polynucleotide complementary to the polynucleotide encoding Mtrm polypeptides. Oligomers may also be employed under less stringent conditions for detection or quantification of closely related DNA or RNA sequences.

A variety of protocols for measuring Mtrm polypeptides, including ELISAs, RIAs, and FACS, are known in the art and provide a basis for diagnosing altered or abnormal levels of Mtrm polypeptides expression. Methods that may also be used to quantify the expression of Mtrm polypeptides include radiolabeling or biotinylating nucleotides, coamplification of a control nucleic acid, and interpolating results from standard curves. (See, e.g., Melba, P. C. et al. (1993) J. Immunol. Methods 159:235-244; Duple, C. et al. (1993) Anal. Biochem. 212:229-236.).

A kit for detecting a Mtrm gene or related nucleotide sequence can be formed. The kit will preferably have a container and a nucleotide sequence, which includes any Mtrm gene or SEQ ID NO 1. The kit is a hybridization kit. The kit should also include a standard or control.

A kit for detecting the allelic state of a Mtrm related gene can be formed. Such a kit will consist of a container and a set of PCR primers spanning the Mtrm related gene in question. The kit will preferably include the necessary components to conduct PCR. The kit will preferably include options for sequencing the PCR products. In addition, such a kit will contain a positive control consisting of a nucleic acid molecule representing a wild-type allele of the gene in question. For example, SEQ ID NO 1, the Mtrm cDNA, or a genomic clone spanning the Mtrm gene may be used as the control.

A kit for detecting a Mtrm protein or polypeptide can also be formed. The kit will preferably have a container and a purified antibody preparation specifically recognizing the Mtrm protein or the corresponding mutant form of a Mtrm related protein in question. Proteins known to react with the antibody will be included as a positive control for such procedures. For example, the polypeptide derived from SEQ ID NO 2 would serve such a purpose.

### DEFINITIONS

Achiasmate refers to meiosis without chiasmata. In those species in which crossing over is limited to one sex, the achiasmate meiosis generally occurs in the heterogametic sex.

Allele refers to a shorthand form of allelomorph, one of a series of possible alternative forms of a given gene (cistron, q.v.), differing in DNA sequence, and affecting the functioning of a single product (RNA and/or protein). If more than two alleles have been identified in a population, the locus is said to show multiple allelism.

The term antibody refers to intact immunoglobulin molecules as well as to fragments thereof, such as Fab, F(ab′)₂, and Fv fragments, which are capable of binding an epitopic determinant. Antibodies that bind to polypeptides of the invention can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term antigenic determinant refers to that region of a molecule (i.e., an epitope) that makes contact with a particular antibody. When a protein or a fragment of a protein is used to immunize a host animal, numerous regions of the protein may induce the production of antibodies that bind specifically to antigenic determinants (particular regions or three-dimensional structures on the protein). An antigenic determinant may compete with the intact antigen (i.e., the immunogen used to elicit the immune response) for binding to an antibody.

The term biologically active refers to a protein having structural, regulatory, or biochemical functions of a naturally occurring molecule. Likewise, “immunologically active” or “immunogenic” refers to the capability of the natural, recombinant, or synthetic Mtrm polypeptide, or of any oligopeptide thereof, to induce a specific immune response in appropriate animals or cells and to bind with specific antibodies.

Bloomington deficiency kit is a group of heterozygotes used to identify genes, which when mutagenized to produce at least one mutant allele, are dosage dependent.

Chiasmata are defined as chromosomal sites where crossing over produces an exchange of homologous parts between nonsister chromatids.

Conservative amino acid substitutions are those substitutions that do not abolish the ability of a subject polypeptide to participate in the biological functions as described herein. Typically, a conservative substitution will involve a replacement of one amino acid residue with a different residue having similar biochemical characteristics such as size, charge, and polarity versus non polarity. A skilled artisan can readily determine such conservative amino acid substitutions.

Degenerate code is one in which a variety of symbols or groups of letters code each different word. The genetic code is said to be degenerate because more than one nucleotide triplet codes for the same amino acid.

Deficiency in cytogenetics refers to the loss of a microscopically visible segment of a chromosome. In a structural heterozygote (containing one normal and one deleted chromosome), the nondeleted chromosome forms an unpaired loop opposite the deleted segment when the chromosomes pair during meiosis.

A Degenerative variant is a protein that has substantially the same function as the wild type protein, but has a different amino acid sequence, where the sequence difference results from the degenerate code.

Dosage dependent relates to an amount of protein that when reduced as a result of heterozygote formation produces a mutant effect.

Drosophila melanogaster species, commonly called the “fruit fly” is a model organism for the study of specific genes in multicellular development and behavior. Its haploid genome contains about 165 million nucleotide pairs. Of these, about 110 million base pairs are unique sequences, present in the euchromatin. The fruit fly is thought to contain about 15,000 genes, and about 1,000 have been cloned. The transcripts range in size from 0.3-13 thousand pairs.

A gene is a hereditary unit that, in the classical sense, occupies a specific position (locus) within the genome or chromosome; a unit that has one or more specific effects upon the phenotype of the organism; a unit that can mutate to various allelic forms; a unit that recombines with other such units. Three classes of genes are now recognized: (1) structural genes that are transcribed into mRNAs, which are then translated into polypeptide chains, (2) structural genes that are transcribed into rRNA or tRNA molecules, which are used directly, and (3) regulatory genes that are not transcribed, but serve as recognition sites for enzymes and other proteins involved in DNA replication and transcription.

Haplo-, when followed by a symbol designating a particular chromosome, indicates an individual whose somatic cells lack one member of the designated chromosome pair. Thus, for example, in Drosophila, haplo-IV means a fly that is monosomic for chromosome 4.

Haplo-insufficiency is defined as a chromosome pair, whereby one allele is mutated.

Haplosis is defined as the establishment of the gametic chromosome number by meiosis.

Heterozygote is a diploid or polyploid individual that has inherited different alleles at one or more loci and, therefore, does not breed true.

Homozygote is an individual or cell characterized by homozygosity, which is the condition of having identical alleles at one or more loci in homologous chromosome segments.

A host organism is an organism that receives a foreign biological molecule, including an antibody or genetic construct, such as a vector containing a gene.

Homology describes the degree of similarity in nucleotide or amino acid sequences between individuals of the same species or among different species. As the term is employed herein, such as when referring to the homology between either two proteins, polypeptides or two nucleotide sequences, homology refers to molecules having substantially the same function, but differing in sequence. Most typically, the two homologous molecules will share substantially the same sequence, particularly in conserved regions, and will have sequence differences in regions of the sequence that does not impact function.

Meiosis, in most sexually reproducing organisms, the doubling of the gametic chromosome number, which accompanies syngamy, is compensated for by a halving of the resulting zygotic chromosome number at some other point during the life cycle. These changes are brought about by a single chromosomal duplication followed by two successive nuclear divisions. The entire process is called meiosis, and it occurs during animal gametogenesis or sporogenesis in plants. The prophase stage is much longer in meiosis than in mitosis. It is generally divided into five consecutive states: leptonema, zygonema, pachynema, diplonema, and diakinesis. When the stages are used as adjectives, the nema suffix is changed to tene.

A mutant is defined as an organism bearing a mutant gene that expresses itself in the phenotype of the organism.

Nondisjunction is defined as the failure of homologous chromosomes (in meiosis I, primary nondisjunction) or sister chromatids (in meiosis II, secondary nondisjunction; or mitosis) to separate properly and to move to opposite poles. Nondisjunction results in one daughter cell receiving both and the other daughter cell none of the chromosomes in question.

Polymorphic restriction fragment is defined as used in Drosophila.

A P element is defined as a transposable element.

Transposable Elements are defined as a class of DNA sequences that can move from one chromosomal site to another. This movement requires a transposase and a resolvase that recognize short nucleotide sequences that are repeated in inverted order at both ends of the element. Transposable elements are also known as “controlling elements.” Transposable elements have been found in yeast, Drosophila caenorhabditis, and humans.

Transposons are defined as one kind of transposable element in both prokaryotes and eukaryotes that is immediately flanked by inverted repeat sequences, which, in turn, are immediately flanked by direct repeat sequences. Transposons usually possess genes in addition to those needed for their insertion (e.g., genes for resistance to antibiotics, sugar fermentation, etc.)

A vector is a self-replication DNA molecule that transfers a DNA segment to a host cell.

Wild type is the most frequently observed phenotype, or the one arbitrarily designated as “normal”. Often symbolized by “+” or “WT.”

A zygote ” is the diploid cell resulting from the union of the haploid male and female gametes.

### EXAMPLES

### Example 1

As will be shown, a protein was identified that is required in normal dosage for hetrochromatic associations to assure the proper segregation of achiasmate chromosomes, the protein was named matrimony (Mtrm). The method of identification was initiated based on the assumption that a reduction in the quantity of some essential ‘glue’ or ‘binder’ protein by 50% might have dramatic and direct effects on achiasmate segregation. A deficiency heterozygote was used, whereby one allele was wt, and the other was a nonfunctional mutant. Resultantly, the expression of a lesser amount of functional protein occurred because one allele expressed a mutant protein.

To start, a Bloomington “deficiency kit” was screened for deficiencies that exhibited a dominant effect on X or 4th chromosomal nondisjunction in females of the genotype FM7/X; Df/+; spa^{pol} (chromosomal balancer 7, X chromosome, deficiency heterozygous, recessive mutation spa allele pol). Genotype abbreviations will be used throughout; an explanation of fly genotype abbreviations is found at www.flybase.com. Attention was focused on deficiencies that impaired the segregation of both achiasmate X and 4th chromosomes, and for which it would be possible to subsequently demonstrate a direct effect on 4th chromosome nondisjunction in females bearing achiasmate X chromosomes.

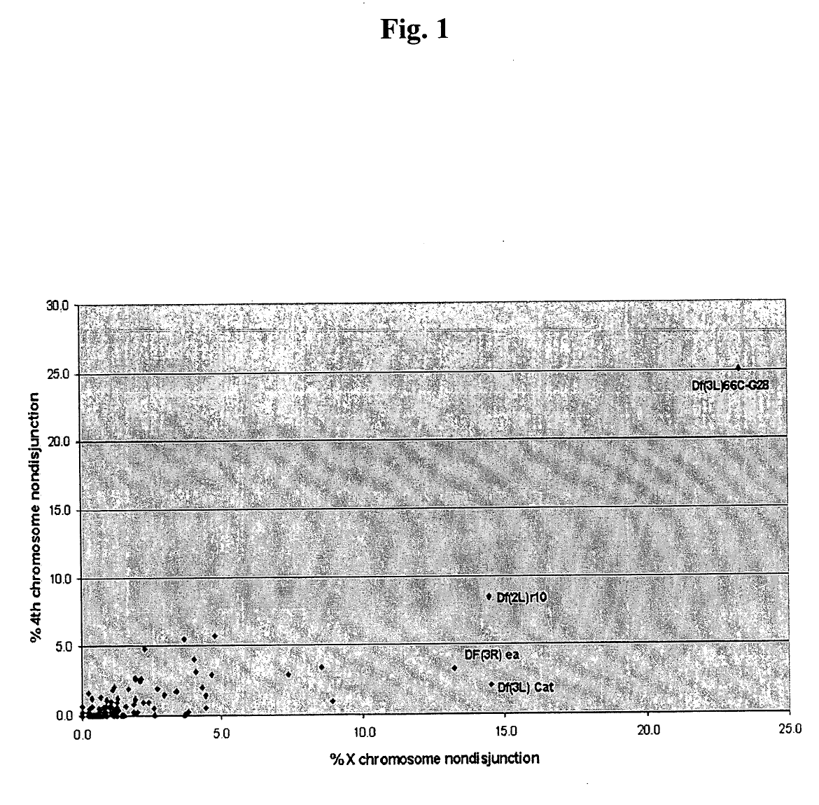

123 autosomal deficiencies obtained from the Bloomington Drosophila Stock Center was screened for a resultant effect on achiasmate segregation. This included 66 second chromosome deficiencies and 57 third chromosome deficiencies. An estimate of the autosomal genome coverage represented by this deficiency collection was approximately 64-73%. The effects of heterozygosity for each of these 123 deficiencies on X and 4th chromosome nondisjunction in FM7/X; Df/+; spa^{pol} females is listed in Table 1. In Table 1, the N column relates to the number of specimens examined. The % X NDJ and % 4 NDJ relates to the nondisjunction percentage for X and 4th chromosomes. This is further illustrated by FIG. 1.

The method was initiated by developing a protocol that eliminated the consideration of genes with additional loci in the screen. First, each deficiency stock was out-crossed to an isogenic lab stock for at least one generation before being tested, and then 10-20 separate single pair matings were performed for each deficiency. Each set of deficiency crosses were examined for obvious indications of heterogeneity that might reflect the activity of a factor capable of segregating from the deficiency. Second, once a candidate region was identified, multiple overlapping deficiencies were tested to establish whether additional deficiencies could phenocopy the original deficiency. By testing overlapping deficiencies spanning the uncovered region, it was possible to both confirm that the observed effect was due to the deficiency and to narrow the interacting genomic interval.

The cross scheme developed introduced marked X and 4th chromosomes. Males from each of the second or third chromosome deficiency stocks were crossed to y w; Sp¹ Bl¹ L^{rm} Bc1 Pu²/SM6a; spa^{pol} or y w; D/TM3; spa^{pol}, respectively. To screen for haplo-insufficient meiotic loci, deficiency bearing males resulting from these crosses were crossed to FM7w; spa^{pol} females to generate y w/FM7w; Df/+; spa^{pol} females. For each deficiency stock, progeny was scored from at least 20 such y w/FM7w; Df/+; spa^{pol} females, crossed individually to attached-XY, y⁺ vfB; C(4), ci ey^{R} males. The frequency of X and 4th chromosome nondisjunction was assessed. (The symbol “attached-XY” denotes the chromosome C(1;Y), IN(1)EN, whose structure is Y^{S}XY^{L}.) Regular male progeny were white Bar (FM7, w/0) and yellow white (yw/0). All regular progeny carried three copies of chromosome 4, the attached-4 chromosome (C(4)RM) and a maternal 4th chromosome marked with spa^{pol}, and were wild type for spa^{pol}, ci and ey^{R}.

As might have been expected, haplo-insufficient loci were few in number. Only four of the deficiencies (Df(3L)66C-G28, Df(2L)r10, Df(3L)Cat, and Df(3Rea) exhibited a statistically significant dominant effect on achiasmate chromosome nondisjunction. The highest frequencies of achiasmate chromosome nondisjunction were exhibited by females heterozygous for Df(3L)66C-G28. Similar high frequencies of X and 4th chromosomal nondisjunction were also exhibited by three smaller deficiencies overlapping the Df(2L)r10 region. None of the three smaller deficiencies that overlap the Df(2L)r10 region: (Df(2L)osp29;Df(2L)TE35Bc-24; or Df(2I)H20) exerted a dominant effect on chromosome segregation. This might suggest the presence of a dosage sensitive region in the interval that is removed only by Df(2L)r10(35F-36A). The gene c(2)M, which encodes a synaptonemal complex component, lies within this interval; however, heterozygosity for a strong allele of this gene did not elevate the frequency of X and 4th chromosomal nondisjunction in FM7/X females (data not shown). Similarly, the meiotic effects of Df(3L)Cat region were not exhibited by the overlapping deficiency Df(3L)W4. This suggested the presence of a dosage sensitive region in the interval that is removed only by Df(3L)Cat(75C-D). There are no obvious candidate genes in this interval. Df(3R)ea, the weakest of the four putative haplo-insufficient enhancers, was not studied further. Thus, it was decided that Df(3L)66C-G28, which showed X and Y disjunction at 23% and 25%, respectively, should be further analyzed.

### Example 2

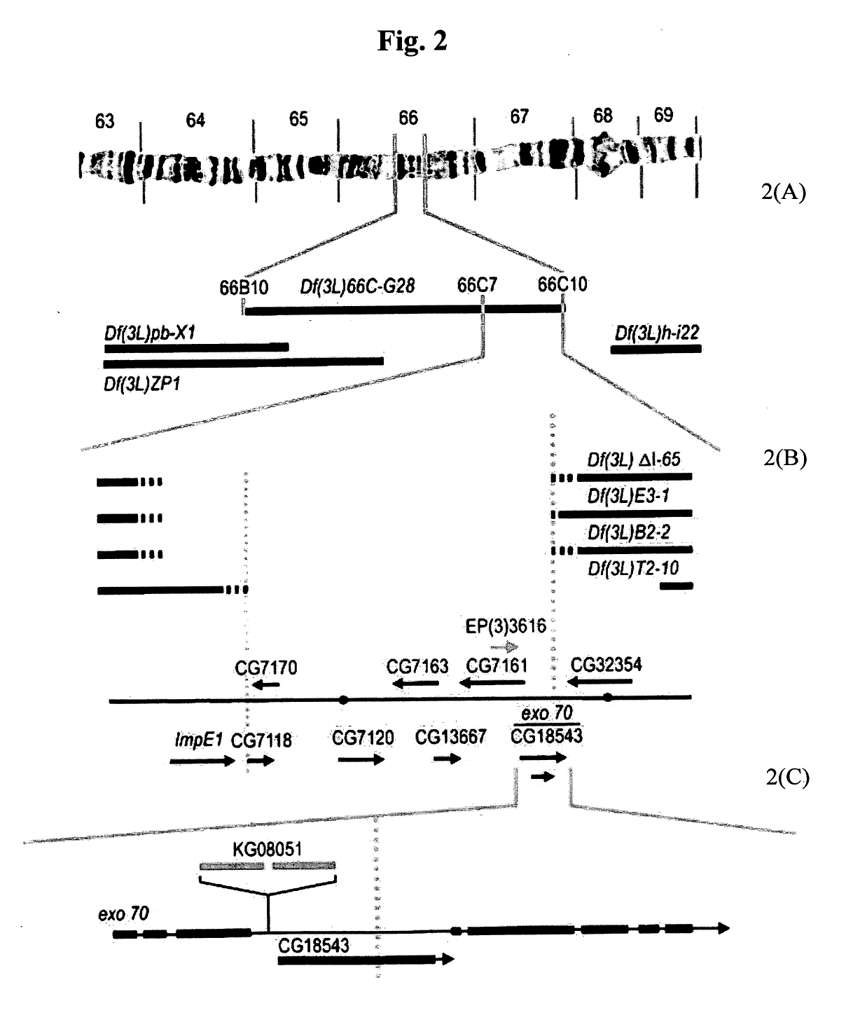

As shown in FIG. 2, the limits of the haplo-insufficient region were established by five deficiencies that both overlapped Df(3L)66C-G28 and also exhibited a dominant defect in the achiasmate chromosome. Df(3L)66C-G28 reached all (or most) of region 66C. The mapping of the breakpoints of these deficiencies on the physical map of the Drosophila genome restricted the putative haplo-insufficient meiotic gene to a small interval within 66C (i.e. the distal and proximal limits of the haplo-insufficient region lie at positions 8,350,000 and 8,372,000, respectively, on the Release 3.1 sequence).

Five deficiencies located with Df(3L)66C-G28 were further examined. The effects of Df(3L)66C-G28 were phenocopied by some, but not all, overlapping deficiencies. The cytological breakpoints of Df(3L)66C-G28 were 66B8-9 and 66C9-10. As shown in Table 2, three overlapping and/or adjacent deficiencies, Df(3L)pbl-X1, Df(3L)ZPI, and Df(3L)h-i22 did not show meiotic defects and, thus, restricted the putative dosage sensitive locus to the interval 66B10; 66C9-10. Four small deficiencies, Df(3L)66C-165, Df(3L)T2-10 and two DEB generated stocks, Df(3L)B2-2 and Df(3L)E3-1, produced a dominant achiasmate chromosome disjunction defect similar to that observed in heterozygotes for Df(3L)66C-G28. These four deficiencies mapped the haplo-insufficient meiotic gene to a small interval within 66C7-10, as shown in FIG. 2.

Table 3 reports a careful examination of the segregational effects of the four deficiencies on both X and 4th chromosome segregation. For both deficiencies, X and 4th chromosome nondisjunction occurred simultaneously far more frequently than would be predicted by chance and most cases of simultaneous X and 4th chromosome nondisjunction are due to XX<->44 segregation. It was noted that most cases of 4th chromosome nondisjunction that occur in the absence of X chromosome misbehavior result in diplo-4 ova.

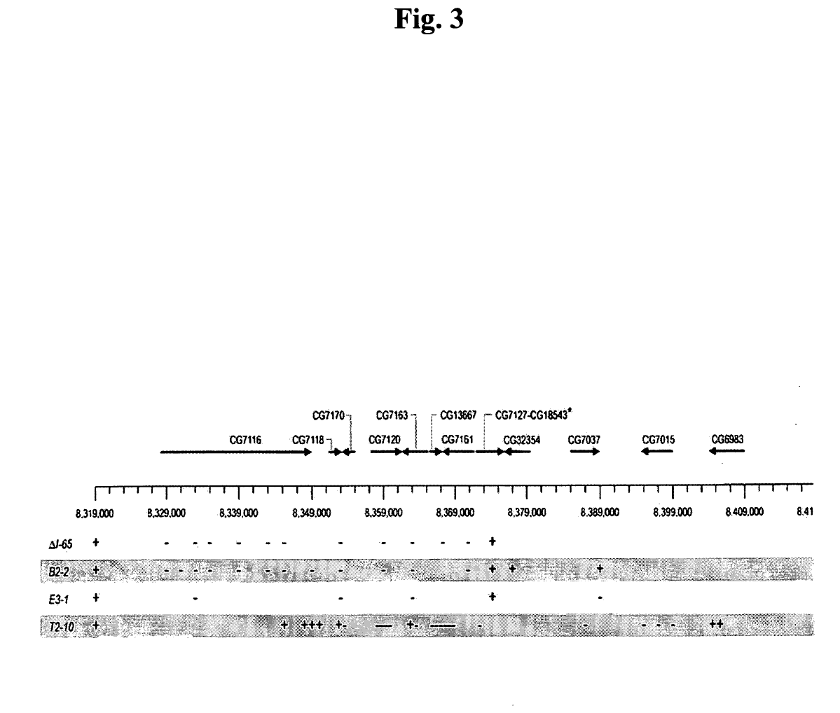

In addition, a fifth deficiency Df(3L) 66C-BSC13 was tested, uncovering the region (66B 12-C 1;66D2-4). This deficiency was produced by recombination between two P elements located at positions 66B10-11 and 66D6 on the map presented in FIG. 3 and resulted in FM7/X; Df/+ females displaying frequencies of X and 4th chromosome nondisjunction of 50.8% and 11.9%, respectively, as shown in Table 2. The fact that this fully independently derived deficiency shared no known background elements in common with the other tested deficiencies confirms that the effects of these deficiencies are a consequence of the deletion of some element in region 66C7-10.

These results pinpointed the region of the gene that caused dosage dependent nondisjunction.

### Example 3

Next, the deficiency breakpoints were characterized. In order to determine the limits of the region that included the putative dosage-sensitive meiotic gene, the position of the ends of the deficiencies were portrayed in FIG. 3 on the physical map of the fly genome. This was accomplished by PCR analysis of homozygous deficiency-bearing oocytes using pairs of primers selected to define specific intervals in the proximal region of 66C. For Df(3L)66C-165, Df(3L)B2-2, and Df(3L)E3-1, the distal breakpoints were proximal to CG7176 (isocitrate dehydrogenase) and were distal to CG1116 (ImpE1). For Df(3L)T2-10, the distal breakpoint was immediate proximal to CG1116 (ImpE1). Thus, the distal limit of the region containing the haplo-insufficient gene was approximated to between 8,310,000 and 8,312,000 on the Flybase map.

For Df(3L)T2-10, the proximal breakpoints were immediately distal to or within CG7015, which is consistent with the failure of these deficiencies to complement lethal alleles of the Cbl gene (CG7037). For Df(3L)B2-2, the proximal breakpoint appears to lie immediately distal to or within the CG718543 (exo70) gene. The breakpoints of Df(3L)66C-165 and Df(3L)E3-1 are more ambiguous and appear to lie between those of Df(3L)B2-2 and Df(3L)T2-10. Nonetheless, the proximal limit of the region containing the haplo-insufficient gene may be approximated to between 8,350,000 and 8,372,000 on the Flybase map.

### Example 4

After identifying the gene region, which exhibited a strong effect on achiasmate segregation, P insertions were used to identify the specific gene that, when eliminated, caused nondisjunction of chromosomes X and 4. The insertions tested are listed in Table 4.

To identify the haplo-insufficient locus, a number of mutations were tested that fall within the 66C 10-11 interval for their ability to mimic the effects of the tested deficiencies with respect to achiasmate nondisjunction. The first mutations to be tested were I(3)F6, I(3)J1, I(3)L3852, EP(3)3729, and EP(3)3616. None of these mutants increased the level of X or 4th chromosomal nondisjunction in FM7/X females above that observed in controls (data not shown).

As shown in Table 4, a P insertion designated P(SUPor-P)KG08051 fully mimicked the effects of deficiencies for 66C in terms of its dominant effect on achiasmate segregation. Df(3L)T2-10 was included for comparison. As shown in FIG. 3, the P(SUPor-P)KG08051 insertion falls into the second intron of the exo70 gene, which encodes a component of a secretory structure known as the exocyst. A second predicted gene (CG18543) also falls within this intron. The insertion site for P(SUPor-P)KG08051 was 90 bp upstream of the predicted start codon for CG18543.

To verify that the KG08051 insertion was the basis of the segregational defect, both precise and imprecise excisions of this P insertion were created. P(SUPor-P)KG08051-exc21 was a precise excision of the P element, as verified by DNA sequencing. This excision did not cause a defect in achiasmate segregation. No X or 4th chromosomal nondisjunctional offspring were obtained among the 450 progeny of FM7/y w; exc24/+ females. Similarly, the P(SUPor-P)KG08051-exc32 derivative, retained only 30 bps of the original P(SUPor-P) insertion, and lost the ability to induce meiotic nondisjunction in FM7/y w; exc32/+ females. (Only one X chromosomal nondisjunctional offspring was obtained among the 612 progeny of FM7/y w; exc32/+females). However, as shown in Table 4, the derivative denoted P(SUPor-P)KG08051-exc 13, which is associated with a large deletion of material upstream of the P insertion (removing the first and second exons of exo70) retained the ability to produce high levels of nondisjunction. The P(SUPor-P)KG08051 insertion exhibited recessive female sterility (associated with defects in oogenesis) and this sterility was alleviated in the precise excision (exc21) previously described.

The P(SUPor-P)KG0805 1 -exc43 excision derivative, which retained the sterility defect, but lost the dominant meiotic effect, allowed separation of the two defects. Although P(SUPor-P)KG08051-exc43 was associated with the loss of both the y⁺ and w⁺ markers, it retained at least the two ends of the P element. Thus, this derivative, may well be the result of an internal deletion that was caused by the loss of the y+ and w+ markers while maintaining a substantial amount of the original sequence. Regardless of the exact nature of the lesion born by this derivative, the fact that it reverted only one of the two phenotypes exhibited by the original insertion, is consistent with the view that the two types of defects may result from the disruption of transcription unit CG18543 by the P(SUPor-P)KG08051 insertion.

The meiotic defects of P(SUPor-P)KG08051 were largely ameliorated in females that also carried a transgenic construct bearing a CG18543 cDNA construct expressed under the control of a UAS promoter and driven by nanos-GAL4. This demonstrated that CG18543 was a dosage sensitive locus that was revealed in the original deficiency screen and that CG18543 exerted its effects in the female germline. The CG18543 transcription unit encoded a predicted protein of 217 amino acids. The CG18543 transcription unit was named matrimony (Mtrm).

It was observed that the hemizygosity for Mtrm exerted its effect on meiosis via a specific impairment of achiasmate segregation. It was determined that hemizygosity for Mtrm had a ten-fold stronger effect on the frequency of X nondisjunction in FM7/X females than it did in females bearing two structurally normal (and thus usually chiasmate) X chromosomes. Second, as might be expected for a defect in achiasmate segregation that directly impairs partner maintenance, hemizygosity for Mtrm exerted a strong effect on 4th chromosome segregation, that was independent of the effect on achiasmate X chromosomes.

### Example 5

If the effects of hemizygosity for Mtrm reflected an impairment of achiasmate segregation, a decrease in the effect on X nondisjunction in females bearing structurally normal (and, thus, usually chiasmate) X chromosomes should be observed. This theory was tested as recited herein.

As shown in Table 5, Df(3L)66C-T2-10, P(SUPor-P)KG08051, and P(SUPor-P)KG08051-exc 13 were tested in females bearing two normal sequence X chromosomes. In all three cases, the levels of X chromosome nondisjunction observed in X/X females were approximately ten-fold lower than the frequencies of X nondisjunction observed in FM7/X females. Moreover, the observed frequencies of X chromosome nondisjunction in females carrying two normal sequence X chromosomes (˜4%) were roughly 40% of the predicted frequency of nonexchange X bivalents (˜8-10%, Hawley et al. 1993), consistent with the view that even these low frequencies of nondisjunction arise as a consequence of the failed segregation of achiasmate X chromosomal bivalents. These observations suggest that hemizygosity for Mtrm primarily, if not exclusively, affects the segregation of nonexchange chromosomes.

A defect in achiasmate segregation that directly impairs partner maintenance would also be expected to exert a strong effect on 4th chromosome segregation that is independent of the effect on achiasmate X chromosomes. As shown in Table 5, the effects of hemizygosity for Mtrm on chromosome 4 nondisjunction are similar in both X/X females and FM7/X females demonstrating a direct effect of Mtrm hemizygosity on 4th chromosome nondisjunction.

The Mtrm hemizygotes are similar to other meiotic mutants affecting achiasmate segregation, in that simultaneous X, 4 nondisjunctional events usually occur more often than might be expected by chance and most cases of simultaneous nondisjunction result from XX<->44 segregations. The excess of simultaneous exceptions and the preponderance of such exceptions that involve XX<->44 segregations both likely reflect the function of a homology-independent third system of meiotic chromosome segregation. This process, referred to as the non-homologous achiasmate segregation does not depend on the physical association of segregational partners, and may simply reflect the ‘crowding’ of spindle poles by one pair of mis-segregating homologs forcing the remaining pair to choose the alternative pole.

### Example 6

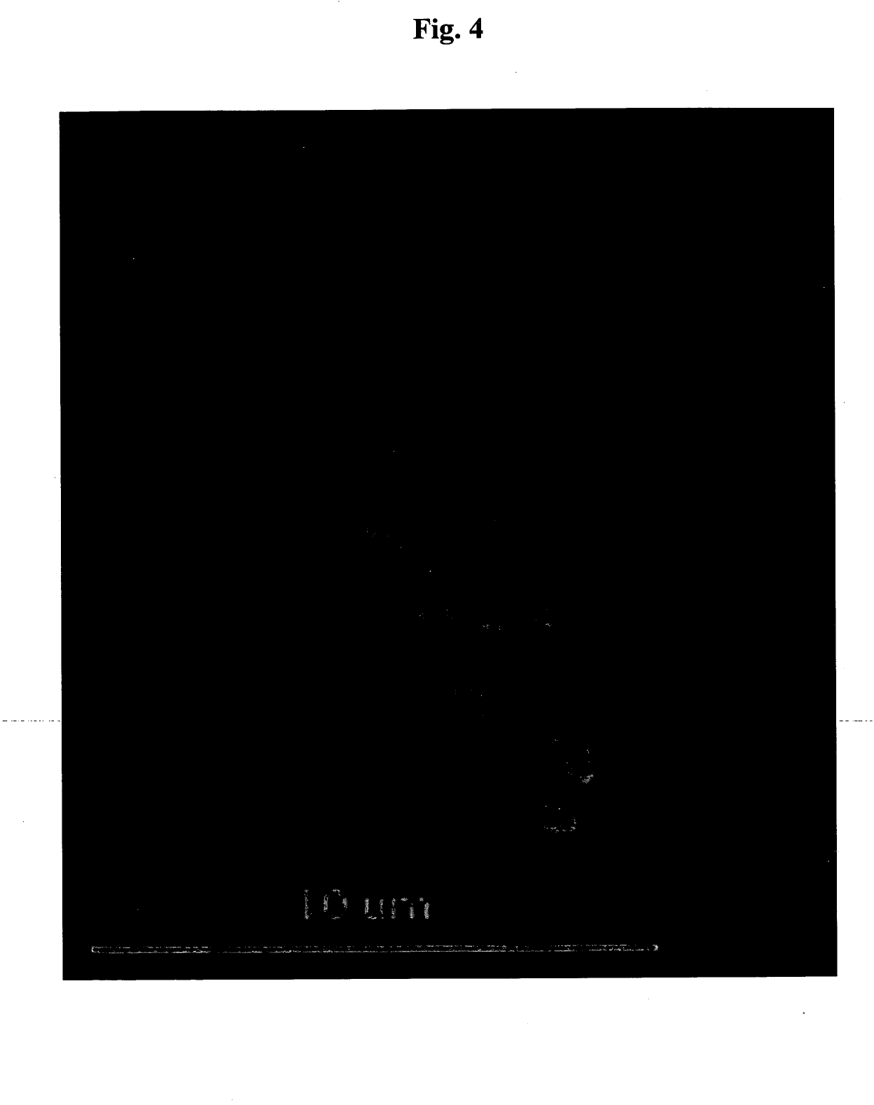

Cytological studies confirmed a specific defect in the position of achiasmate chromosomes on the meiotic spindles of oocytes produced by FM7/X; Df(3L)66C-T2-10 females (i.e. females that are hemizygous for Mtrm). The vast majority of prometaphase and metaphase figures observed in these oocytes were normal bipolar spindles, suggesting that these oocytes were competent to assemble proper bipolar spindles. However, as exemplified in FIG. 4, achiasmate X and 4th chromosomes were often improperly placed on metaphase 1 spindles of these oocytes from females.

Cytological studies of prometaphase and metaphase one in deficiency bearing or haplo-insufficient females demonstrated that assembly of a bipolar meiotic spindle occurred normally in greater than 90% of the oocytes. Further, the achiasmate chromosomes remained associated with the spindle in all cases observed; however, achiasmate chromosomes appeared to be randomly positioned on either side of the metaphase mass, with respect to the position of the homolog. One possible interpretation of this data is the presence of a single copy of a site or gene in region 66C that disrupts the presence or functioning of whatever “glue” holds paired homologs together from SC breakdown at the end of pachytene until metaphase I.

Among a sample of 19 metaphase figures in which the achiasmate X and 4th chromosomes were clearly discernable, the achiasmate X chromosomes are found on the same half-spindle in 21% (4/19) of the oocytes, and the 4th chromosomes are found on the same half-spindle in 31% of the oocytes (6/19). In two of the cases of X chromosome nondisjunction, the two X chromosomes were on one half spindle and the two 4th chromosomes were on the other, indicative of XX<->44 segregation events. Two cases were observed involving the 4th chromosome, and one involving the X chromosome, in which although the achiasmate homologs were found on opposite halves of the spindle, they were not observed to be on the same arc of the spindle. Thus, it seems unlikely that these spindles reflect the segregation of the two homologs from their partners. Rather, they likely reflected the chance orientation of the two homologs to opposite poles. X or 4th chromosomes dissociated from the spindle were not observed. Indeed, the only obvious defect was the lack of proper positioning of achiasmate chromosomes on the meiotic spindle of a large fraction of the oocytes examined.

### Example 7

In the cytological studies of Example 6, the oocytes were prepared and examined, as follows: Egg chambers from 3-7 day old females were extracted by quick pulses of a blender using a modified Robb's medium. The mixture was passed sequentially through a loose and fine mesh to separate late stage oocytes. The oocytes were fixed for 5 minutes on a rotator at room temperature in a hypertonic solution, therefore preventing hypotonic activation of the mature oocytes. After removal of the follicle cells, chorion and vitelline membranes, the oocytes were permeabilized with 1% Triton X-100 in PBS. Oocytes were labeled with YL1/2 (1:200) and YOL 1/34 (1:200) rat anti-tubulin monoclonal antibodies (Accurate). Both Oligreen (Molecular Probes 1:10,000) and 1 MAB52 (1:500) anti-core histone mouse monoclonal primary antibodies were then labeled with secondary antibodies (1:250) purchased from Jackson Immunoresearch conjugated in the following manner: Cy2 to anti-mouse, and Cy3 to anti-rat<-. Oocytes were examined using a DeltaVision Deconvolution Microscope.

### Example 8

A kit can be prepared, which includes a container, which holds an isolated nucleotide sequence, such as SEQ ID NO 1. The isolated nucleotide sequence can be used to form cDNA probes, which are included in the kit. The probes will also include labels, which will be inserted according to known procedures. The DNA sample can then be obtained from a non-human animal. This sample can be tested to determine if it is homologous to SEQ ID NO 1 by using the kit. The sample DNA obtained from a non-human animal will be contacted with SEQ ID NO 1 probes in the container of the kit. If the sample DNA attaches to the probe, it will indicate the presence of a gene analog in the non-human mammal. This would indicate a high likelihood that achiasmate disjunction is dosage dependent.

### Example 9

A kit can be prepared, which includes a container, which holds an isolated wild-type nucleic acid molecule, such as SEQ ID NO 1. This isolated nucleic acid molecule can be used to form a cDNA probe, which is included in the kit. The probe will also include a label, which will be inserted according to known procedures. The DNA sample can then be obtained from a non-human animal. This sample can then be tested to determine if it is homologous to SEQ ID NO 1 by using the kit. The sample DNA obtained from a non-human animal will be contacted with the SEQ ID NO 1 probe in the container of the kit. If the sample DNA attaches to this probe, it will indicate the presence of a wild-type gene analog in the non-human animal.

Thus, there has been shown and described a gene and method related to haplo-insufficiency in achiasmate segregation which fulfills all the objects and advantages sought therefor. It is apparent to those skilled in the art, however, that many changes, variations, modifications, and other uses and applications to the gene and method related to haplo-insufficiency in achiasmate segregation are possible, and also such changes, variations, modifications, and other uses and applications which do not depart from the spirit and scope of the invention are deemed to be covered by the invention, which is limited only by the claims which follow.

### REFERENCE LIST

Afshar, K., Scholey, J., and Hawley, R.S. 1995. Identification of the chromosome localization domain of the Drosophila Nod kinesin-like protein. J Cell Biol 131(4): 833-843.
Afshar, K., Barton, N., Hawley, R. S., and Goldstein, L. S. B. 1995. DNA binding and meiotic chromosomal localization of the Drosophila Nod kinesin-like protein. Cell 81(1): 129-138.
Arbeitman et al. 2002. Gene expression during the life cycle of Drosophila melanogaster. Science 297(5590): 2270-2275.
Carpenter, A. T. C. 1973. A meiotic mutant defective in distributive disjunction in Drosophila melanogaster. Genetics 73(3): 393-428.
Carpenter, A. T. C. 1979. Synaptonemal complex and recombination nodules in wild-type Drosophila melanogaster females. Genetics 92: 511-541.
Carpenter, A. T. C. 2003. Normal synaptonemal complex and abnormal recombination nodules in two alleles of the Drosophila melanogaster mutant mei-W68. Genetics 163: 1337-1356.
Celniker, S. E. et al. 2002. Finishing a whole-genome shotgun: Release 3 of the Drosophila melanogaster euchromatic genome sequence. Genome Biol. 3(12): Research 0079-9.
Dernburg, A. F., Sedat, J. W., and Hawley, R. S. 1996. Direct evidence of a role for hetrochromatin in meiotic chromosome segregation. Cell 86: 135-146.
Edgar, 1993. Drosophila melanogaster Sevelin clone D52 cell cycle arrest protein gene, complete cds. GenBank/EMBLIDDBJ: U03288.
Grell, R. F. 1976. Distributive Pairing Segregation in: M. Ashburner and E. Novitski, eds. The Genetics and Biology of Drosophila. Volume 1a. Academic Press, London, pp. 436-486.
Guinta, K. L., Jang, J. K., Manheim, E. A., Subramanian, G., and McKim, K. S. 2002. subito Encodes a Kinesin-like Protein Required for Meiotic Spindle Pole Formation in Drosophila melanogaster. Genetics 160(4): 1489-1501.
Halsell, S. R., and Kiehart, D. P. 1998. Second-site noncomplementation identifies genomic regions required for Drosophila nonmuscle myosin function during morphogenesis. Genetics 148(4): 1845-1863.
Harris, D., Orme, C., Kramer, J. J., Namba, R. Champion, M. D., Palladino, M.J., Natzle, J. E., (matrimonh) that is haplo-insufficient for achiasmate segregation in Drosophila oocytes Genetics (in press).
Hawley, R. S. and Theurkauf. 1993. Requiem for the distributive system: Achiasmate segregation in Drosophila females. Trends in Genetics 9: 310-317.
Hawley, R. S. 1988. Exchange and chromosomal segregation in eukaryotes. In Genetic Recombination. G. Smith and R. Kucherlapati, eds. ASM Press, Washington, D.C. pp. 497-527.
Hawley, R. S., Irick, H., Zitron, A. E., Haddox, D. A., Lohe, A., New C., Whitley, M., Arbel, T., Jang, J., McKim, K., and Childs, G. C. 1993. There are two systems of achiasmate disjunction in Drosophila, one of which requires heterochromatic homology. Developmental Genetics 13: 440-467.
Hinton, C. W. 1966. Enhancement of recombination associated with the c3G mutant of Drosophila melanogaster. Genetics 53(1): 157-164.
Karpen, G. H., Le, M. H., and Le, H. 1996. Centric hetrochromatin and the efficiency of achiasmate disjunction in Drosophila female meiosis.
Kimble, M., and Church, K. 1983. Meiosis and early cleavage in Drosophila melanogaster eggs: effects of the claret nondisjunctional mutation. J. Cell Sci. 62, 301-318.
Komma, D. J., Home, A. S., and Endow, S. A. 1991. Separation of meiotic and mitotic effects of claret nondisjunctional on chromosome segregation in Drosophila. EMBO J. 10(2): 419-424.
Kramer, J. J. and Hawley, R. S. 2003. Mutants in the spindle-associated transmembrane protein Axs impair meiotic spindle assembly in Drosophila oocytes. Nature Cell Biology 5: 261-263.
Lee, L. A., Elfring, L. K., Bosco, G., and Orr-Weaver, T. L. 2001. A genetic screen for suppressors and enhancers of the Drosophila PAN GU cell cycle kinase identifies cyclin B as a target. Genetics 158(4): 1545-1556.
Lindsley, D. L. and Sandler, L. 1977. The genetic analysis of meiosis in female Drosophila melanogaster. Phil. Trans R Soc Lond. Series B. 277: 295-312.
Manheim, E. A. and McKim, K. S. 2003. The Synaptonemal Complex Component C(2)M Regulates Meiotic Crossing over in Drosophila. Curr Biol. 13(4): 276-285
Matthies, H. J., McDonald, H. B., Goldstein, L. S., and Theurkauf, W. E. 1996. Anastral meiotic spindle morphogenesis: role of the non-claret disjunctional kinesin-like protein. J. Cell Bio. 134, 455-464.
Matthies, H. J. G., Clarkson, M., Saint, R. B., Namba, R., and Hawley, R. S. 2000. Analysis of meiosis in fixed and live oocytes by light microscopy. Drosophila: Protocols. Eds. W. S. Sullivan, M. Ashburner, and R. S. Hawley. Cold Spring Harbor Press, N.Y. pp. 67-85.
Matthies, H. J., et al. 1999. Mutations in the alpha-tubulin 67C gene specifically impair achiasmate segregation in Drosophila melanogaster. J Cell Biol 147(6): 1137-1144.
Matthies, H. J., Baskin, R. J., and Hawley, R. S. 2001. Orphan Kinesin NOD Lacks Motile Properties But Does Possess a Microtubule-stimulated ATPase Activity. Molec. Biol. of the Cell. 12(12): p. 4000-4012.
McKim, K. S., Green-Marroquin, B., Sekelsky, J. J., Chin, G., Steinberg, C., Khodosh, R., and Hawley, R. S. 1998. Meiotic synapsis in the absence of recombination. Science 279: 876-878.
Murphy, T. D. and Karpen, G. H. 1995. Localization of centromere function in a Drosophila minichromosome. Cell 82: 599-609.
Murthy, M., Garza, D., Scheller, R. H., and Schwarz, T. L. 2003. Mutations in the exocyst component Sec5 disrupt neuronal membrane traffic, but neurotransmitter release persists. Neuron. 37(3): 433-447.
Nicklas, R. B. 1974. Chromosome segregation mechanisms. Genetics 78(1): 205-213.
O'Tousa, J., 1982. Meiotic chromosome behavior influenced by mutation-altered disjunction in Drosophila melanogaster females. Genetics 102: 503-524.
Page, J., Berrios, S., Rufas, J. S., Parra, M. T., Suja, J. A., Heyting, C., and Femandez-Donoso, R. 2003. The pairing of X and Y chromosomes during meiotic prophase in the marsupial species Thylamys elegans is maintained by a desne plate developed from their axial elements. J Cell Sci. 116: 551-560.
Rasmussen, S. W. 1977. The transformation of the synaptonemal complex into the elimination chromatin of Bombyx mori oocytes. Chromosoma 60: 205-221.
Rasooly, R. S., New, C. M., Zhang, P., Hawley, R. S., and Baker, R. S. 1991. The lethal(1)TW-6^{cs} mutation of Drosophila melanogaster is a dominant antimorphic allele of nod, and is associated with a single base change in the putative ATP-binding domain. Genetics 129: 409-422.
Rasooly, R. S., Zhang, P., Tibolla, A. K., and Hawley, R. S. 1994. A structure-function analysis of NOD, a kinesin-like protein from Drosophila melanogaster. Mol. and Gen. Genetics 242, 145-151.
Robbins, L. G. 1977. The meiotic effect of a deficiency in Drosophila melanogaster with a model for the effects of enzyme deficiency on recombination. Genetics 87(4): 655-684.
Robbins, L. G. 1980. Matemal-zyogotic lethal interactions in Drosohpila melanogaster: the effects of deficiencies in the zeste-white region of the X chromosome. Genetics 96(1): 187-200.
Robbins, L. G. 1981. The meiotic behavior of some single-cistron mutants in the zeste-white region of the Drosophila melanogaster X chromosome. Mol Gen Genet 183(2): 214-219.
Sandler and Novitski, E. 1956. Evidence for genetic homology between chromosomes I and IV in Drosophila melanogaster, with a proposed explanation for the crowding effect in triploids. Genetics 41: 189-193.
Sekelsky, J. J., McKim, K. S., Arbel, T., Chin, G., French, R. L., Hari, K. L., Hurley, W., Jang, J. K., Laurencon, A., Madden, L., Messina, L., Milliken, D., Page, S. D., Ring, A. D., Wayson, S., Zimmerman, C., and Hawley, R. S. 1999. Identification of novel meiotic genes recovered in a P element screen. Genetics 152: 529-542.
Theurkauf, W. E. and Hawley, R. S. 1992. Meiotic spindle assembly in Drosophila females:

behavior of nonexchange chromosomes and the effects of mutations in the Nod kinesin-like protein. J Cell Biol 116(5): 1167-1180.
vanDoren, M., Williamson, A. L., and Lehmann, R. 1998. Regulation of zygotic gene expression in Drosophila primordial germ cells. Curr Biol. 8(4): 243-246.
Wald, H. 1936. Cytological studies on the abnormal development of the eggs of the claret mutant type of Drosophila simulans. Genetics 21: 264-279.
Wasser, M. and Chia, W. 2003. The Drosophila EAST protein associates with a nuclear remnant during mitosis and constrains chromosome mobility. J Cell Sci. 116(Pt9): 1733-1743.
Whyte, W., Irick, H., Arbel, T., Yasuda, G., French, R., Falk, D. R., and Hawley, R. S. 1993. The genetic analysis of distributive pairing in Drosophila melanogaster. III. The wildtype product of the Axs locus is required for achiasmate meiotic segregation. Genetics 134: 825-833.
Wolf, K. W. 1994. How meiotic cells deal with non-exchange chromosomes. Bioessays 16(2): 107-114.
Zhang, P. and Hawley, R. S. 1990. The genetic analysis of distributive segregation in Drosophila melanogaster. II. Further genetic analysis of the nod locus. Genetics 125: 115-127.

Zitron, A. E. and Hawley, R. S. 1989. The genetic analysis of distributive segregation in Drosophila melanogaster. I. Isolation and characterization of Aberrant X segregation, Axs, a mutant defective in chromosome partner choice. Genetics 122: 801-821.

TABLE 1
Effects of the tested deficiencies on X and 4th chromosome nondisjunction.

| Deficiency Name and Breakpoints | | N | % X NDJ | % 4 NDJ |
|---|---|---|---|---|
| Df(2L)PMF | 21A1; 21B7-8 | 929 | 0.4 | 0.0 |
| Df(2L)a1 | 21B8-C1; 21C8-D1 | 153 | 2.6 | 0.0 |
| Df(2L)S2 | 21C6-D1; 22A6-B1 | 777 | 0.3 | 0.5 |
| Df(2L)dp79b | 22A2-3; 22D5-E1 | 155 | 1.3 | 0.0 |
| Df(2L)C144 | 23A1-4; 23C3-5 | 351 | 2.3 | 0.0 |
| In(2LR)DTD16LDTD42R (Df(2L)) | 23C; 23E3-6 | 812 | 7.4 | 3.0 |
| Df(2L)JS32, dppd-ho | 23C3-5; 23D1-2 | 1124 | 0.4 | 0.0 |
| Df(2L)S2590, P{ry[+t7.2]}FK1 | 23D2; 23E3 | 1968 | 0.6 | 0.5 |
| Df(2L)edSz-1 | 24A3-4; 24D3-4 | 1224 | 0.5 | 0.0 |
| Df(2L)GpdhA | 25D7-E1; 26A8-9 | 276 | 8.0 | 2.2 |
| Df(2L)E110 | 3C1-2; 20F; 25F3- | 767 | 2.6 | 0.5 |
| | 26A1; 26D3-11 | | | |
| Df(2L)J136-H52 | 27C2-9; 28B3-4 | 633 | 1.3 | 0.6 |
| Df(2L)spd | 27D-E; 28C | 899 | 2.0 | 0.2 |
| Df(2L)wgCX3 | 27F2; 28A | 480 | 4.6 | 3.3 |
| Df(2L)Trf-C6R31 | Within 28DE | 821 | 1.7 | 1.9 |
| Df(2L)N22-5 | 29C3-5; 30C4-D1 | 531 | 0.4 | 0.0 |
| Df(2L)N22-14 | 29C1-2; 30C8-9; 30D1- | 132 | 1.5 | 0.0 |
| | 2; 31A1-2 | | | |
| Df(2L)30C | 29F7-30A1; 30C2-5 | 473 | 2.1 | 2.5 |
| Df(2L)S1402, P{w[+mc] = lacw}1402 | 30C1-2; 30F, 30B9-10 | 949 | 1.1 | 1.1 |
| Df(2L)Pr1 | 32F1-3; 33F1-2 | 391 | 1.5 | 1.5 |
| Df(2L)esc10 | 33A8-B1; 33B2-3 | 823 | 0.0 | 0.2 |
| Df(2L)osp29 | 35B1-3; 35E6 | 271 | 3.7 | 0.0 |
| Df(2L)TE35Bc-24 | 35B4-6; 35F1-7 | 376 | 6.4 | 0.8 |
| Df(2L)TE35B-4 | 35C1-35D5 | 360 | 0.6 | 0.0 |
| Df(2L)r10 | 35D1-2; 36A6-7 | 401 | 14.5 | 8.5 |
| Df(2L)H20(d12034) | 36A8-9; 36E1-2 | 141 | 2.8 | 0.0 |
| Df(2L)TW137 | 36C2-4; 37B9-C1 | 414 | 1.0 | 0.0 |
| Df(2L)pr76 | 37D; 38E | 864 | 0.2 | 0.0 |
| Df(2L)E55 | 37D2-E1; 37F5-38A1 | 339 | 3.5 | 2.4 |
| Df(2L)TW84 | 37F5-38A1; 39D3-E1 | 492 | 3.3 | 0.4 |
| Df(2L)C′ | 40H35; 40h38L | 1113 | 0.7 | 0.0 |
| Df(2R)nap1 | 41D2-E1; 42B1-3, 41A- | 253 | 4.0 | 1.6 |
| | B; 42BC | | | |
| Df(2R)cn88b | 42A; 42E | 1218 | 1.1 | 0.7 |
| Df(2R)St1 | 42B3-5; 43E15-18 | 525 | 1.9 | 0.8 |
| Df(2R)pk78s | 42C1-7; 43F5-8 | 720 | 1.1 | 0.6 |
| | (42B; 42Cmax) | | | |
| Df(2R)cn9 | 42E; 44C | 634 | 1.3 | 0.9 |
| Df(2R)H3C1 | 43F; 44D3-8 | 748 | 1.9 | 0.8 |
| Df(2R)44CE | 44C4-5; 44E2-4 | 344 | 8.1 | 3.5 |
| Df(2R)H3E1 | 44D1-4; 44F12 | 470 | 0.9 | 0.0 |
| Df(2R)Np5 | 44F10; 45D9-E1, 31B; | 969 | 0.6 | 0.2 |
| | 45D9-E1 | | | |
| Df(2R)w45-30n | 45A6-7; 45E2-3 | 700 | 2.3 | 4.9 |
| Df(2R)B5 | 46A; 46C | 610 | 0.7 | 0.0 |
| Df(2R)X1 | 46C; 47A1 | 300 | 2.0 | 0.0 |
| Df(2R)stan2 | 46F1-2; 47D1-2 | 331 | 6.6 | 10.9 |
| Df(2R)E3363 | 47A; 47F | 590 | 0.3 | 0.7 |
| Df(2R)en = A | 47D3; 48A5-6 | 1113 | 0.9 | 0.0 |
| Df(2R)en30 | 48A3-4; 48C6-8 | 809 | 0.2 | 0.5 |
| Df(2R)vg135 | 48D-E; 49D-E | 732 | 2.7 | 1.9 |
| Df(2R)CB21 | 48E; 49A | 799 | 1.3 | 0.5 |
| Df(2R)trix | 51A1-2; 51B6 | 626 | 2.2 | 1.0 |
| Df(2R)Jp1 | 51C3; 52F5-9 | 329 | 1.2 | 0.0 |
| Df(23R)Jp8 | 52F5-9; 52F10-53A1 | 591 | 0.7 | 1.4 |
| Df(2R)PcP7B | 54E8-F1; 55B9-C1 | 1260 | 1.4 | 1.4 |
| Df(2R)Pc111B | 54F6-55A1; 55C1-3 | 242 | 0.8 | 0.8 |
| Df(2R)PC4 | 55A; 55F | 622 | 4.8 | 5.8 |
| Df(2R)AA21 | Df56F9-17; 57D11-12, | 940 | 1.1 | 1.1 |
| | In 38E; 56E | | | |
| Df(2R)59AD | 59A01-03; 59D01-D04 | 520 | 1.5 | 0.0 |
| Df(2R)or-BR6 | 59D05-10; 60B03-08, | 275 | 1.5 | 0.0 |
| | 40; 60E04[L]40F; 59E[R] | | | |
| Df(2R)Chi[g230] | 60A3-7; 60B4 | 776 | 0.8 | 0.5 |
| Df(2R)Px2 | 60C5-6; 60D9-10 | 1024 | 0.8 | 0.6 |
| Df(2R)Px2 | 60C5-6; 60D9-10 | 1024 | 0.8 | 0.6 |
| Df(2R)M60E | 60E6-9; 60E11 | 1199 | 0.5 | 0.0 |
| Df(2R)Kr10 | 60E10; 60F5 | 991 | 2.4 | 1.0 |
| Df(3L)emc-E12 | 61A; 61D3 | 601 | 0.7 | 0.0 |
| Df(3L)Aprt-1 | 62A10-B1; 62D2-5 | 1513 | 1.2 | 2.1 |
| Df(3L)R | 62B7; 62B12 | 1161 | 4.5 | 0.5 |
| Df(3L)R-G7 | 62B8-9; 62F2-5 | 1850 | 1.9 | 1.2 |
| Df(3L)HR232 | 63C1; 63D3 | 954 | 1.0 | 0.2 |
| Df(3L)HR119 | 63C6; 63E | 1580 | 1.9 | 0.3 |
| Df(3L)GN24 | 63F4-7; 64C13-15 | 245 | 13.1 | 7.3 |
| Df(3L)XD198 | 65A2; 65E1 | 1436 | 0.8 | 0.3 |
| Df(3L)pbl-X1 | 65F3; 66B10 | 1044 | 1.3 | 0.4 |
| Df(3L)66C-G28 | 66B8-9; 66C9-10 | 1797 | 23.3 | 25.0 |
| Df(3L)h-i22 | 66D10-11; 66E1-2 | 587 | 0.0 | 0.0 |
| Df(3L)SWcf-R6 | 66E1-6; 66F1-6 | 1320 | 1.2 | 0.5 |
| Df(3L)Rd1-2 | 66F5 | 840 | 0.2 | 0.2 |
| Df(3L)Ixd6 | 67E1-2; 68C1-2 | 893 | 0.4 | 0.0 |
| Df(3L)vin5 | 68A2; 69A1 | 784 | 3.8 | 0.3 |
| Df(3L)vin7 | 68C8-11; 69B4-5 | 167 | 7.2 | 3.6 |
| Df(3L)iro-2 | 691-5; 69D1-6 | 819 | 8.5 | 3.4 |
| Df(3L)Ly | 70A2-3; 70A5-6 | 784 | 4.3 | 2.0 |
| Df(3L)fzGF3b | 70/b?; 70D6 | 1163 | 1.0 | 0.7 |
| Df(3L0fz[D21] | 70D; 71F | 1058 | 1.1 | 0.0 |
| Df(3L)BK10 | 71C; 71F | 471 | 0.4 | 0.0 |
| Df(3L)st-f13 | 72C1-D1; 73A3-4 | 515 | 1.9 | 2.7 |
| Df(3L)st[e4] | 72D5-10; 73A5 | 558 | 1.1 | 0.7 |
| Df(3L)W4 | 75B8-11; 75C5-7 | 1226 | 0.7 | 0.3 |
| Df(3L)Cat | 75B8; 75F1 | 482 | 14.5 | 2.1 |
| Df(3L)VW3 | 76A3; 76B2 | 677 | 0.9 | 0.0 |
| Df(3L)rdgC | 77A1; 77D1 | 1130 | 2.3 | 3.4 |
| Df(3L)ri79C | 77B-C; 77F-78A | 893 | 0.9 | 1.1 |
| Df(3L)Pc-2q | 78B-C; 77F-78A | 893 | 0.9 | 1.1 |
| Df(3L)Delta 1AK | 79F; 80A | 893 | 1.1 | 0.4 |
| Df(3R)ME15 | 81F3-6; 82F5-7 | 1367 | 0.6 | 0.0 |
| Df(3R)w[11118]; Df(3R)6-7 | 82D3-8; 82F3-6 | 1433 | 4.5 | 1.4 |
| Df(3R)3-4 | 82F3-4; 82F10-11 | 1647 | 4.1 | 3.2 |
| Df(3R)Tp110, Dp(3; 3)Dfd[rvX1] | 83C1-2; 84B1-2, 83D4- | 1095 | 1.1 | 0.5 |
| | 5; 84A4-5; 98F1-2 | | | |
| Df(3R)Scr | 84A1-2; 84B1-2 | 1164 | 0.2 | 0.5 |
| Df(3R)pXT103 | 84F14; 85C-D | 832 | 8.9 | 1.0 |
| Df(3R)by10 | 85D8-12; 85E7-F1 | 276 | 0.7 | 0.0 |
| Df(3R)M-Kx1 | 86C1; 87B1-5 | 263 | 2.3 | 0.0 |
| Df(3R)ry615 | 87B11-13; 87E8-11 | 926 | 0.9 | 0.6 |
| Df(3R)su[Hw] | 88A9-B2 | 543 | 0.4 | 0.0 |
| Df(3R)red[p52] | 88A12-B1; 88B4-5 | 1167 | 3.4 | 1.7 |
| Df(3R)red1 | 88B1; 88D3-4 | 621 | 1.6 | 0.6 |
| In(3R)Vbx[P18]/Df(3)R red P931 | 88A10-B1; C2-3 | 571 | 1.1 | 0.7 |
| Df(3R)ea | 88E7-13; 89A1 | 1573 | 13.2 | 3.3 |
| Df(3R)P14 | 90C2-D1; 91A1-2 | 806 | 1.5 | 0.5 |
| Df(3R)Cha7 | 90F1-4; 91F5 | 544 | 1.1 | 1.8 |
| Df(3R)H-B79 | 92B3; 92F13 | 503 | 2.4 | 2.4 |
| Df(3R)23D1 | 93D; 94F | 406 | 3.0 | 3.0 |
| Df(3R)hhE23 | 94A; 95 | 866 | 0.2 | 1.6 |
| Df(3R)crb-F89-4 | 95D7-D11; 95F15 | 213 | 1.9 | 0.0 |
| Df(3R)crb87-5 | 95F7; 96A17-18 | 400 | 3.0 | 1.5 |
| Df(3R)96B | 96A21; 96C2 | 226 | 2.7 | 0.0 |
| Df(3R)T1-P | 97A; 98A1-2 | 108 | 3.7 | 5.6 |
| Df(3R)D605 | 97E3; 98A5 | 577 | 0.0 | 0.7 |
| Df(3R)3450 | 98E3; 99A6-8 | 188 | 0.0 | 0.0 |
| Df(3R)Dr-rv1 | 99A1-2; 99B6-11 | 1150 | 0.0 | 0.0 |
| Df(3R)faf-BP | 100D; 100F5 | 779 | 1.0 | 0.5 |

TABLE 2
Evidence for a gene in region 66C that is haplo-insufficient
for achiasmate chromosome segregation

| | % nondisjunction | | |
|---|---|---|---|
| | X | 4th | Ad- |
| Cytological | Chromo- | Chromo- | justed |
| Breakpoints | some | some | Total |

a)y w/FM7, w; Df(3L)/+

| | | | | |
|---|---|---|---|---|
| Df(3L)66C-G28 | 66B8-9; 66C9-10 | 23.3 | 25.0 | 1797 |
| Df(3L)pbl-X1 | 65F3; 66B10 | 1.3 | 0.0 | 1044 |
| Df(3L)h-i12 | 66D10; 66E1-2 | 0.0 | 0.0 | 587 |
| Df(3L)ZP1 | 66A17; 66C5 | 1.0 | 0.7 | 602 |

b)y w/FM7, w; Df(3L)66C+

| | | | | |
|---|---|---|---|---|
| Df(3L)66C-G28 | 66B8-9; 66C9-10 | 23.3 | 25.0 | 1797 |
| Df(3L)66CI65 | 66C7-10 | 33.9 | 10.4 | 1250 |
| Df(3L)66C-B2-2 | 66C7-10 | 31.3 | 6.7 | 1458 |
| Df(3L)66C-E3-1 | 66C7-10 | 38.1 | 30.1 | 714 |
| Df(3L)66C-T2-10 | 66C7-10 | 39.2 | 27.7 | 1307 |
| Df(3L)66C-BSC13 | 66B12-C1; 66D2-4 | 50.8 | 11.9 | 751 |

TABLE 3
Variation in levels of X and 4th chromosome nondisjunction for four deficiencies in 66C

| | | | % nondisjunction | | |
|---|---|---|---|---|---|
| | Cytological Breakpoints | | X Chromosome | 4th Chromosome | Adjusted Total |
| DF(3L)66C-G28 | 66B8-9; 66C9-10 | Experiment 1 | 23.3 | 25.0 | 1797 |
| | | Experiment 2 | 25.8 | 12.7 | 883 |
| Df(3L)66C-I65 | 66C7-10 | Experiment 1 | 33.9 | 10.4 | 1250 |
| | | Experiment 2 | 21.5 | 11.1 | 1461 |
| | | 18° | 24.9 | 23.7 | 587 |
| | | 25° | 17.4 | 21.2 | 665 |
| | | 30° | 13.7 | 22.8 | 702 |
| Df(3L)66C-B2-2 | 66C7-10 | Experiment 1 | 17.4 | 5.2 | 688 |
| | | Experiment 2 | 31.3 | 6.7 | 1458 |
| Df(3L)66C-T2-10 | 66C7-10 | 18° | 38.7 | 24.1 | 517 |
| | | 25° | 39.2 | 27.7 | 1307 |
| | | 30° | 31.6 | 24.4 | 664 |

TABLE 4
Detailed segregational effects of heterozygosity for KG08051 and its derivatives. FM7/X; spapol females for each
indicated P element insertion were crossed to appropriate tester males (see Materials and Methods) to allow the
assessment of both X and 4th chromosome nondisjunction. Data for Df(3L)T2-10 are provided for comparison.

| Oocyte Genotype | Paternal enotype | +* | Df(3L)T2-10 | KG0801** | Exc13 | Exc21 | Exc43 | P{lacW}MP1 |
|---|---|---|---|---|---|---|---|---|
| Normal | | | | | | | | |
| X; 4 | XY; 44 | 8115 | 293 | 137 | 101 | 274 | 360 | 456 |
| X; 4 | O; 44 | 6063 | 290 | 140 | 89 | 182 | 254 | 370 |
| X nondisj. | | | | | | | | |
| 0; 4 | XY;44 | 10 | 109 | 41 | 47 | 0 | 3 | 1 |
| XX; 4 | O; 44 | 10 | 72 | 36 | 48 | 0 | 1 | 2 |
| 4 nondisj. | | | | | | | | |
| X; O | XY; 44 | 12 | 41 | 25 | 14 | 0 | 1 | 0 |
| X; O | O; 44 | 2 | 12 | 7 | 6 | 0 | 1 | 0 |
| X; 44 | XY; O | 2 | 86 | 11 | 52 | 0 | 1 | 0 |
| X; 44 | O; O | 10 | 73 | 7 | 11 | 0 | 2 | 0 |
| X; 4 nondisj. | | | | | | | | |
| O; O | XY; 44 | 1 | 6 | 3 | 4 | 0 | 2 | 0 |
| XX; 44 | O; O | 0 | 5 | 2 | 5 | 0 | 0 | 0 |
| O; 44 | XY; O | 0 | 30 | 46 | 18 | 0 | 0 | 0 |
| XX: O | O; 44 | 0 | 34 | 34 | 9 | 0 | 0 | 0 |
| Total progeny | | 14225 | 1051 | 488 | 404 | 456 | 625 | 829 |
| Adjusted Total | | 14246 | 1307 | 488 | 404 | 456 | 625 | 829 |
| % nullo-X | | 0.1 | 22.1 | 27.7 | 25.8 | 0.0 | 1.6 | 0.2 |
| % diplo-X | | 0.1 | 16.9 | 22.1 | 23.2 | 0.0 | 0.3 | 0.5 |
| Total % X | | | | | | | | |
| Nondisjunction | | 0.2 | 39.2 | 49.8 | 49.0 | 0.0 | 1.9 | 0.7 |
| % nullo-4 | | 0.1 | 10.1 | 16.3 | 8.6 | 0.0 | 1.0 | 0.0 |
| % diplo-4 | | 0.1 | 17.5 | 16.0 | 20.3 | 0.0 | 0.4 | 0.0 |
| Total % 4 | | | | | | | | |
| Nondisjunction % | | 0.2 | 27.7 | 32.3 | 29.0 | 0.0 | 1.4 | 0.0 |
| Nonhomologous | | | | | | | | |
| Segregations | | | 8.1 | 23.0 | 6.7 | | | |

*Control data are from Zitron and Hawley (1989)
**See Materials and Methods

TABLE 5
Comparison of the segregational effects of heterozygosity for Df(3L)66C-
T2-10, KG08051, and KG08051-exc13 in FM7/X; spa^{pol} and X/X; spa^{pol} females.

| Oocyte | Paternal | FM7/X; | X/X; | FM7/X; | X/X; | FM7/X; | |
|---|---|---|---|---|---|---|---|
| Genotype | enotype | Df(3L)T2-10 | Df(3L)T2-10 | KG08051 | KTG08051 | Exc13 | X/X; Exc13 |
| Normal | | | | | | | |
| X; 4 | XY; 44 | 293 | 148 | 137 | 375 | 101 | 374 |
| X; 4 | O; 44 | 290 | 192 | 140 | 475 | 89 | 413 |
| X nondisj. | | | | | | | |
| O; 4 | XY; 44 | 109 | 2 | 41 | 8 | 47 | 6 |
| XX; 4 | O; 44 | 72 | 2 | 36 | 4 | 48 | 7 |
| 4 nondisj. | | | | | | | |
| X; O | XY; 44 | 41 | 33 | 25 | 43 | 14 | 72 |
| X; O | O; 44 | 12 | 29 | 7 | 58 | 6 | 51 |
| X; 44 | XY; O | 86 | 68 | 11 | 77 | 52 | 104 |
| X; 44 | O; O | 73 | 107 | 7 | 101 | 11 | 128 |
| X: 4 nondisj. | | | | | | | |
| O; O | XY; 44 | 6 | 1 | 3 | 4 | 4 | 0 |
| XX; 44 | O; O | 5 | 3 | 2 | 4 | 5 | 3 |
| O; 44 | XY; O | 30 | 2 | 46 | 4 | 18 | 2 |
| XX; O | O; 44 | 34 | 2 | 34 | 2 | 9 | 0 |
| Total Progeny | | 1051 | 589 | 488 | 1155 | 404 | 1160 |
| Adjusted Total | | 1307 | 601 | 650 | 1181 | 535 | 1178 |
| % nullo-X | | 22.1 | 1.7 | 27.7 | 2.7 | 25.8 | 1.4 |
| % diplo-X | | 16.9 | 2.3 | 22.1 | 1.7 | 23.2 | 1.7 |
| Total % X | | 39.2 | 4.0 | 49.8 | 4.4 | 49.0 | 3.1 |
| nondisj. | | | | | | | |
| % nullo-4 | | 10.1 | 11.3 | 16.3 | 9.5 | 8.6 | 10.4 |
| % diplo-4 | | 17.5 | 30.8 | 16.0 | 16.4 | 20.3 | 20.5 |
| Total % 4 | | 27.7 | 42.1 | 32.3 | 26.0 | 29.0 | 31.0 |
| nondisj. % non- | | 8.1 | | 23.0 | | 6.7 | |
| homologous | | | | | | | |
| segregations | | | | | | | |
| 018538/064993 | | | | | | | |
| KJDOT 260308 | | | | | | | |

What is claimed is:

1. An isolated nucleotide sequence comprising a sequence that encodes a polypeptide having the amino acid sequence of SEQ ID NO. 2, or of a fragment of SEQ ID NO. 2 that is at least 15 amino acid residues in length.

2. The isolated nucleotide sequence of claim 1, wherein the sequence encodes a polypeptide that is able to mediate the disjunction of achiasmate chromosomes in a dose dependent manner.

3. An isolated nucleotide sequence comprising a sequence that encodes a polypeptide having SEQ ID NO. 2, or SEQ ID NO. 2 with conservative amino acid substitutions.

4. The isolated nucleotide sequence of claim 3, wherein the sequence encodes a polypeptide that is able to mediate the disjunction of achiasmate chromosomes in a dose dependent manner.

5. An isolated nucleotide sequence comprising a sequence that encodes a polypeptide the amino acid sequence of which is at least 50% identical to the amino acid sequence of SEQ ID NO. 2.

6. The isolated nucleotide sequence of claim 5, wherein the sequence encodes a polypeptide that is able to mediate the disjunction of achiasmate chromosomes in a dose dependent manner.

7. The isolated nucleotide sequence of claim 5, wherein the sequence encodes a polypeptide that is at least 75% identical to the amino acid sequence of SEQ ID NO. 2.

8. The isolated nucleotide sequence of claim 5, wherein the sequence encodes a polypeptide that is at least 90% identical to the amino acid sequence of SEQ ID NO. 2.

9. The isolated nucleotide sequence of claim 5, wherein the sequence encodes a polypeptide that is at least 95% identical to the amino acid sequence of SEQ ID NO. 2.

10. The isolated nucleotide sequence of claim 5, wherein the sequence encodes a polypeptide that is at least 99% identical to the amino acid sequence of SEQ ID NO. 2.

11. An isolated nucleotide sequence comprising a sequence at least 50% identical to SEQ ID NO. 1.

12. The isolated nucleotide sequence of claim 11, wherein the sequence is at least 75% identical to SEQ ID NO. 1.

13. The isolated nucleotide sequence of claim 11, wherein the sequence is at least 90 identical to SEQ ID NO. 1.

14. The isolated nucleotide sequence of claim 11, wherein the sequence is at least 95% identical to SEQ ID NO. 1.

15. The isolated nucleotide sequence of claim 11, wherein the sequence is at least 99% identical to SEQ ID NO. 1.

16. An isolated nucleotide sequence comprising a sequence that hybridizes under stringent conditions to a hybridization probe the nucleotide sequence of which consists of SEQ ID NO.1, or the complement of SEQ ID NO. 1.

17. An isolated nucleotide sequence comprising a sequence that encodes a polypeptide with the amino acid sequence of SEQ ID NO. 2.

18. The isolated nucleotide sequence of claim 17, wherein the sequence encodes a polypeptide that is able to mediate the disjunction of achiasmate chromosomes in a dose dependent manner.

19. An isolated nucleotide sequence comprising the sequence of SEQ ID NO. 1, or a degenerate variant of SEQ ID NO. 1.

20. An isolated nucleotide sequence consisting of the sequence of SEQ ID NO. 1.

21. An isolated nucleotide sequence selected from the group consisting of a homolog, ortholog, degenerate variant, homologous fragment, antisense sequence, and mutant of a sequence having SEQ ID NO 1.

22. The ortholog nucleotide sequence of claim 21, wherein the ortholog nucleotide sequence is derived from the group consisting of vertebrates and invertebrates.

23. The mutant nucleotide sequence of claim 21, wherein the mutation is selected from the group consisting of frame shift, point, and deletion mutations.

24. An isolated Mtrm nucleotide sequence comprising a sequence at least 50% identical to SEQ ID NO. 1, wherein the sequence encodes a polypeptide that is able to mediate the disjunction of achiasmate chromosomes in a dose dependent manner.

25. An expression vector comprising the nucleotide sequence of claim 1 operably linked to an expression control sequence.

26. A cultured cell comprising the expression vector of claim 25.

27. A cultured cell comprising the nucleotide sequence of claim 1 operably linked to an expression control sequence.

28. A cultured cell transfected with the vector of claim 25, or a progeny of the cell, wherein the cell expresses the polypeptide.

29. A method of producing a polypeptide, the method comprising culturing the cell of claim 26 under conditions permitting the expression of the polypeptide.

30. A method of producing a polypeptide, the method comprising culturing the cell of claim 27 under conditions permitting expression under the control of the expression control sequence, and purifying the protein from the cell or the medium of the cell.

31. A transposable element comprising SEQ ID NO 1.

32. A transposable element comprising a nucleotide sequence selected from the group consisting of a homolog, ortholog, degenerate variant, homologous fragment, antisense sequence, and mutant of a sequence having SEQ ID NO 1.

33. A transposable element, comprising a Mtrm nucleotide sequence.

34. The transposable element of claim 33, wherein the transposable element is a P-element.

35. A transposable element comprising a wt Mtrm nucleotide sequence and a mutant Mtrm nucleotide sequence.

36. A heterozygote which causes haplo insufficiency, comprising a wt Mtrm nucleotide sequence and a transfected mutant Mtrm nucleotide sequence.

37. A transfected haplo-insufficient chromosome comprising a mutant Mtrm nucleotide sequence.

38. An isolated heterozygote nucleotide sequence for causing haplo-insufficiency during meiosis, wherein at least one Mtrm allele nucleotide sequence is mutated to form a heterozygous mutant.

39. An isolated nucleotide sequence for identifying haplo-insufficiency in an organism, comprising a mutant Mtrm gene.

40. A purified polypeptide that specifically binds to an antibody that binds specifically to a polypeptide having SEQ ID NO. 2.

41. The purified polypeptide of claim 40, wherein the polypeptide is able to mediate the disjunction of achiasmate chromosomes in a dose dependent manner.

42. A purified polypeptide, the amino acid sequence of which comprises a sequence at least 50% identical to SEQ ID NO. 2.

43. The purified polypeptide of claim 42, wherein the polypeptide is able to mediate the disjunction of achiasmate chromosomes in a dose dependent manner.

44. The purified polypeptide of claim 42, wherein the polypeptide is at least 75% identical to the amino acid sequence of SEQ ID NO. 2.

45. The purified polypeptide of claim 42, wherein the polypeptide is at least 90% identical to the amino acid sequence of SEQ ID NO. 2.

46. The purified polypeptide of claim 42, wherein the polypeptide is at least 95% identical to the amino acid sequence of SEQ ID NO. 2.

47. The purified polypeptide of claim 42, wherein the polypeptide is at least 99% identical to the amino acid sequence of SEQ ID NO. 2.

48. A purified polypeptide, the amino acid sequence of which comprises SEQ ID NO. 2 with 0 to 50 conservative amino acid substitutions.

49. The purified polypeptide of claim 48, wherein the polypeptide is able to mediate the disjunction of achiasmate chromosomes in a dose dependent manner.

50. The purified polypeptide of claim 48, wherein the amino acid sequence of which comprises SEQ ID NO. 2 with 0 to 25 conservative amino acid substitutions.

51. The purified polypeptide of claim 48, wherein the amino acid sequence of which comprises SEQ ID NO. 2 with 0 to 10 conservative amino acid substitutions.

52. A purified immunogenic polypeptide, the amino acid sequence of which comprises at least ten consecutive residues of SEQ ID NO. 2.

53. The polypeptide of claim 52, wherein the polypeptide is able to mediate the disjunction of achiasmate chromosomes in a dose dependent manner.

54. A purified polypeptide, the amino acid sequence of which comprises SEQ ID NO.2.

55. The polypeptide of claim 54, wherein the polypeptide is able to mediate the disjunction of achiasmate chromosomes in a dose dependent manner.

56. A purified polypeptide, the amino acid sequence of which consists of SEQ ID NO.2.

57. The polypeptide of claim 56, wherein the polypeptide is able to mediate the disjunction of achiasmate chromosomes in a dose dependent manner.

58. The polypeptide of claim 54, wherein the polypeptide is derived from vertebrates and invertebrates.

59. An isolated Mtrm polypeptide comprising an amino acid sequence at least 50% identical to SEQ ID NO. 2, wherein the polypeptide is able to mediate the disjunction of achiasmate chromosomes in a dose dependent manner.

60. A purified antibody that binds specifically to a polypeptide having SEQ ID NO. 2.

61. The purified antibody of claim 60, wherein the antibody is a monoclonal or polyclonal antibody.

62. The purified antibody of claim 60, wherein the antibody is a variant selected from the group consisting of a single chain recombinant antibody, a humanized chimeric antibody, a Fab fragment antibody, and a Fab′ fragment antibody.

63. A method of making an antibody, comprising immunizing a non-human animal with an immunogenic fragment of a polypeptide having SEQ ID NO. 2.

64. A method of purifying a polypeptide having SEQ ID NO. 2 from a biological sample containing the polypeptide, the method comprising:
(a) providing an affinity matrix comprising the antibody of claim 60 bound to a solid support;
(b) contacting the biological sample with the affinity matrix, to produce an affinity matrix-polypeptide complex;
(c) separating the affinity matrix-polypeptide complex from the remainder of the biological sample; and
(d) releasing the polypeptide from the affinity matrix.

65. A method for preventing haplo-insufficiency comprising:
(a) using a Mtrm gene to identify a mutant Mtrm gene in an organism;
(b) forming a P-element having a wt Mtrm nucleotide sequence; and,
(c) transfecting the mutant host organism with the P-element.

66. A kit for detecting Mtrm mutants and wild types comprising:
(a) a Mtrm nucleotide sequence; and,
(b) a container.

67. A hybridization kit for detecting a Mtrm mutant gene, wherein the kit comprises:
(a) a container; and,
(b) a nucleotide sequence selected from a group consisting of SEQ ID NO 1, and homologous sequences thereof.

68. A kit for detecting a Mtrm gene comprising:
(a) PCR primers spanning a Mtrm family gene
(b) a positive control; and,
(c) sequencing products.

69. A kit for detecting a Mtrm polypeptide, wherein the kit comprises:
(a) a container; and,
(b) an antibody derived from polypeptide selected from a group consisting of SEQ ID NO 2, and homologs thereof.

70. A method for screening females prior to conception for detection of mutants, comprising:
(a) using a Mtrm gene to identify organisms which have a mutant Mtrm gene;
(b) forming a P-element comprised of SEQ ID NO 1; and,
(c) transfecting the host organism with the P-element.

71. A nucleotide sequence for mutating a Mtrm gene sequence comprising:
(a) a P-element; and,
(b) a mutant Mtrm nucleotide sequence.

72. A method for causing nondisjunction in vivo, comprising:
(a) forming a Mtrm P-element by combining a mutant Mtrm, nucleotide sequence with a P-element; and,
(b) transfecting a host with the Mtrm P-element, sufficient to cause a dosage dependent Mtrm mutant organism.
